(19) United States
(12) Patent Application Publication
(10) Pub. No.: US 2006/0134001 A1
(43) Pub. Date: Jun. 22, 2006

(54) Conjugated infrared fluorescent substances for detection of cell death

(76) Inventor: John V. Frangioni, Wayland, MA (US)

Correspondence Address: FISH & NEAVE IP GROUP;ROPES & GRAY LLP, ONE INTERNATIONAL PLACE, BOSTON, MA, 02110-2624, US

(21) Appl. No.: 10/521,066

(22) PCT Filed: Jul. 10, 2003
(86) PCT No.: PCT/US03/21478

§ 371 Date: Oct. 11, 2005

Related U.S. Application Data
(60) Provisional application No. 60/395,582, filed on Jul. 12, 2002.

(51) Int. Cl. A61K 49/00

(52) U.S. Cl. 424/9.6; 530/409

(57) ABSTRACT

One aspect of the invention is an infrared fluorescent reagent that may be useful to provide images of regions of cell death. The reagent may comprise a fluorescent substance, preferably an infrared fluorescent substance, conjugated to a targeting moiety that selectively localizes or binds to cells or tissue undergoing cell death, such as annexin V. The reagent may also comprise an infrared fluorescent substance associated with an antibody, antibody fragment, or ligant that accumulates within a region of diagnostic significance, such as a region characterized by necrosis or apoptosis. In one embodiment, the reagent comprises annexin V covalently conjugated to IRDye 78. In another embodiment, the reagent comprises annexin V covalently conjugated to a quantum dot. More broadly, the reagents described herein may be used in detecting cell death.

### BACKGROUND OF THE INVENTION

Apoptosis (programmed cell death) and necrosis are two major processes by which cells die. Although comparable in outcome, they are distinctly different processes. Generally, apoptosis is triggered by environmental factors that activate endogenous endonucleases activity. Another difference between the two is the integrity of the cell membrane. Cell membrane integrity is lost at a late stage in apoptosis but is the normal result of necrosis.

Absorption and fluorescent dyes, such as indocyanine green, have proven useful for imaging apoptosis and/or necrosis. Some of the more commonly used dyes share a number of useful characteristics. First, the dyes are suitable for labeling antibodies or low-molecular-weight ligands of diagnostic significance, or can otherwise be adapted for sequestration or preferential uptake at a site of interest such as a lesion. The dyes are safe for injection or other introduction into a live subject. And finally, the dyes emit light at a specific wavelength when excited, so that their location and concentration may be tracked.

However, once administered to a subject, these dyes do not seek out cells undergoing cell death. There remains a need for fluorescent reagents that specifically target apoptotic and/or necrotic tissues.

### SUMMARY OF THE INVENTION

One aspect of the invention is an infrared fluorescent reagent that maybe useful to provide images of regions of cell death. The reagent may comprise a fluorescent substance, preferably an infrared fluorescent substance, such as a dye or other compound, conjugated to a targeting moiety that selectively localizes or binds to cells or tissue undergoing cell death, such as annexin V. The reagent may also comprise an infrared fluorescent substance associated with an antibody, antibody fragment, or ligand that accumulates within a region of diagnostic significance, such as a region characterized by necrosis or apoptosis. In one embodiment, the reagent comprises annexin V covalently conjugated to IRDye78. In another embodiment, the reagent comprises annexin V covalently conjugated to a quantum dot. More broadly, the reagents described herein may be used in detecting cell death.

Exemplary fluorescent substances will emit at wavelengths to which blood and tissue are relatively transparent, such as in the infrared region. Infrared substances are especially useful for these purposes, because an infrared fluorescent substance will not absorb wavelengths that tissues absorb strongly, and they should not have emission wavelengths that will be absorbed by tissues. Accordingly, such infrared radiation leads to enhanced transmission through tissues and allows imaging of the deeper areas of a subject.

### BRIEF DESCRIPTION OF DRAWINGS

The invention will be appreciated more fully from the following further description thereof, with reference to the accompanying drawings, wherein:

FIG. 1 shows an embodiment of an imaging system for use during open surgery;

FIG. 2 shows a near-infrared window used by the imaging system;

FIG. 3 shows an embodiment of an imaging system for use in an endoscopic tool;

FIG. 4 shows an image displaying both a circulatory system and surrounding tissue; and

FIG. 5 depicts the results of an assay using the conjugated annexin V as described herein.

FIGS. 6 and 7 show results of near-infrared fluorescence imaging using labeled annexin V in vivo.

### DETAILED DESCRIPTION OF THE INVENTION

One aspect of the invention is an infrared fluorescent reagent that may be useful to provide images of regions of cell death. The reagent may comprise a fluorescent substance, preferably an infrared fluorescent substance, such as a dye or other chemical compound, conjugated to a targeting moiety that selectively localizes or binds to cells or tissue undergoing cell death, such as annexin V. The reagent may also comprise an infrared fluorescent substance associated with an antibody, antibody fragment, or ligand that accumulates within a region of diagnostic significance, such as a region characterized by necrosis or apoptosis. In one embodiment, the reagent comprises annexin V covalently conjugated to IRDye78. In another embodiment, the reagent comprises annexin V covalently conjugated to a quantum dot. More broadly, the reagents described herein may be used in detecting cell death.

Exemplary fluorescent substances will emit at wavelengths to which blood and tissue are relatively transparent, such as in the infrared region. Infrared substances are especially useful for these purposes, because an infrared fluorescent substance will not absorb wavelengths that tissues absorb strongly, and they should not have emission wavelengths that will be absorbed by tissues. Accordingly, such infrared radiation leads to enhanced transmission through tissues and allows imaging of the deeper areas of a subject.

I. Definitions

The term “cell death” in the context of “detecting cell death” or “localizing cell death” refers to cells that have lost plasma membrane integrity, as well as to the processes by which mammalian cells die. Such processes include apoptosis and processes thought to involve apoptosis (e.g., cell senescence), as well as necrosis. “Cell death” is used herein to refer to the death or imminent death of nucleated cells (e.g., neurons, myocytes, hepatocytes, etc.) as well as to the death or imminent death of anucleate cells (e.g., red blood cells, platelets, etc.).

The term “infrared fluorescent substance” refers to compounds that fluoresce in the infrared region (680 nm to 100,000 nm) of the spectrum, such as near infrared (700 nm to 1000 nm) to mid infrared (1000 nm to 20,000 μm) to far infrared (20,000 nm to 100,000 nm). These substances include iodocyanine green, IRDye78, IRDye80, IRDye38, IRDye40, IRDye41, IRDye700, IRDye800, Cy7, IR-786, DRAQ5NO, quantum dots, and analogs thereof.

“Homology” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. Variants for use in the present invention include peptides or proteins comprising an amino acid sequence at least 60% identical to the identified agent, wherein the variant retains the function of the identified agent (e.g., localizing to an area of cell death). Further examples of variants for use in the present invention include peptides and proteins comprising an amino acid sequence at least 70%, 75%, 80%, 85%, 90%, 95%, or greater than 95% identical to an naturally occurring polypeptide that possesses the desired function, wherein the variant retains the function of the known agent (e.g., the protein localizes to an area of cell death).

Additionally, the invention contemplates the use of bioactive fragments of proteins useful in the subject methods. The term “bioactive fragment” refers to a fragment of contiguous amino acid residues of a suitable protein or polypeptide that retains the function of the full-length protein or polypeptide (e.g., the fragment localizes to an area of cell death). The invention further contemplates the use of variants of bioactive fragments of useful proteins comprising an amino acid sequence at least 60%, 70%, 75%, 80%, 85%, 90%, or greater than 95% identical to a useful bioactive fragment of the protein, wherein the variant of the bioactive fragment retains the function of the identified agent (e.g., the fragment localizes to an area of cell death).

Monodispersed particles are defined as having at least 60% of the particles fall within a specified particle size range. Monodispersed particles deviate less than 10% in rms diameter and preferably less than 5%.

Quantum yield is defined as the ratio of photons emitted to that absorbed.

II. Cell Death—Apoptosis and Necrosis

Apoptosis refers to “programmed cell death” whereby the cell executes a “cell suicide” program. It is now thought that the apoptosis program is evolutionarily conserved among virtually all multicellular organisms, as well as among all the cells in a particular organism. Further, it is believed that in many cases, apoptosis may be a “default” program that must be actively inhibited in healthy surviving cells.

The decision by a cell to submit to apoptosis may be influenced by a variety of regulatory stimuli and environmental factors (Thompson, 1995). Physiological activators of apoptosis include tumor necrosis factor (TNF), Fas ligand, transforming growth factor A, the neurotransmitters glutamate, dopamine, N-methyl-D-aspartate, withdrawal of growth factors, loss of matrix attachment, calcium and glucocorticoids. Damage-related inducers of apoptosis include heat shock, viral infection, bacterial toxins, the oncogenes myc, rel and E1A, tumor suppressor p53, cytolytic T-cells, oxidants, free radicals and nutrient deprivation (antimetabolites). Therapy-associated apoptosis inducers include gamma radiation, UV radiation and a variety of chemotherapeutic drugs, including cisplatin, doxorubicin, bleomycin, cytosine arabinoside, nitrogen mustard, methotrexate and vincristine. Toxin-related inducers of apoptosis include ethanol and β-amyloid peptide.

Apoptosis can have particularly devastating consequences when it occurs pathologically in cells that do not normally regenerate, such as neurons. Because such cells are not replaced when they die, their loss can lead to debilitating and sometimes fatal dysfunction of the affected organ. Such dysfunction is evidenced in a number of neurodegenerative disorders that have been associated with increased apoptosis, including Alzheimer's disease, Parlcinson's disease, amyotrophic lateral sclerosis, retinitis pigmentosa and cerebellar degeneration.

The consequences of undesired apoptosis can be similarly devastating in other pathologies as well, including ischemic injury, such as typically occurs in cases of myocardial infarction, reperfusion injury and stroke. In particular, apoptosis is believed to play a central role in very delayed infarction after mild focal ischemia (Du, et al., 1996). Additional diseases associated with increased apoptosis include, but are not limited to, the following: AIDS; myelodysplastic syndromes, such as aplastic anemia; and toxin-induced liver disease, including damage due to excessive alcohol consumption.

Necrosis is the localized death of cells or tissue due to causes other than apoptosis (i.e., other than the execution of the cell's intrinsic suicide program). Necrosis can be caused by traumatic injury, bacterial infection, acute hypoxia and the like. There is some overlap between the two types of cell death, in that some stimuli can cause either necrosis or apoptosis or some of both, depending on the severity of the injury.

III. Asymmetry of Biological Membranes

It is generally believed that biological membranes are asymmetric with respect to specific membrane phospholipids. In particular, the outer leaflet of eukaryotic plasma membranes is formed predominantly with the cholinephospholipids, such as sphingomyelin and phosphatidylcholine (PC), whereas the inner leaflet contains predominantly aminophospholipids, such as phosphatidylserine (PS) and phosphatidylethanolamine (PE). This asymmetry is thought to be maintained by the activity of an adenosine triphosphate (ATP)-dependent aminophospholipid translocase, which selectively transports PS and PE between bilayer leaflets (Seigneuret and Devaux, 1984). Other enzymes thought to be involved in the transport of phospholipids between leaflets include ATP-dependent floppase (Connor, et al., 1992) and lipid scramblase (Zwaal, et al., 1993).

Although asymmetry appears to be the rule for normal cells, the loss of such asymmetry is associated with certain physiological, as well as pathogenic, processes. For example, it has been recognized that membrane asymmetry, detected as appearance of PS on the outer leaflet of the plasma membrane (“PS exposure”), is one of the earliest manifestations of apoptosis, preceding DNA fragmentation, plasma membrane blebbing, and loss of membrane integrity (Martin, et al., 1995; Fadok, et al., 1992).

Similar re-orientation has been observed in sickle cell disease (Lane, et al., 1994), β-thalassemia (Borenstain-Ben Yashar, et al., 1993), platelet activation, and in some mutant tumor cell lines with defective PS transport. A gradual appearance of PS on the outer leaflet has also been observed to occur in aging red blood cells (Tait and Gibson, 1994). When the PS exposure on such cells reaches a threshold level, the cells are removed from circulation by macrophages (Pak and Fidler, 1991). All of the above conditions proximately culminate in the death of the affected cells (i.e., cells with significant PS exposure).

It will be appreciated that PS exposure is a component in both apoptosis and necrosis. Its role in the initial stages of apoptosis is summarized above. Once the apoptotic cell has reached the terminal stages of apoptosis (i.e., loss of membrane integrity), it will be appreciated that the PS in both plasma membrane leaflets will be “exposed” to the extracellular milieu. A similar situation exists in cell death by necrosis, where the loss of membrane integrity is either the initiating factor or occurs early in the necrotic cell death process; accordingly, such necrotic cells also have “exposed” PS, since both plasma membrane leaflets are “exposed”.

IV. Targeting Moieties

The annexin family of proteins are examples of targeting moieties useful in the practice of the present invention. Annexins are amphiphilic proteins that can bind reversibly to cellular membranes in the presence of cations. The primary structure of the annexins comprises a fourfold or eightfold repeated domain that contains a consensus sequence. Although the physiological function of annexins has not been fully elucidated, several properties of annexins make them useful as diagnostic and/or therapeutic agents. In particular, it has been discovered that annexins possess a very high affinity for anionic phospholipid surfaces, such as a membrane leaflet having an exposed surface of phosphatidylserine (PS).

Recombinant annexin offers several advantages, including ease of preparation and economic efficiency. A number of different annexins have been cloned from humans and other organisms. Their sequences are available in sequence databases, including GenBank Q9UJ72 (human), O76027 (human), P50995 (human), P09525 (human), P27216 (human), P20073 (human), P13928 (human), P08133 (human), Q9QZ10 (mouse), P07356 (mouse), P48036 (mouse), P14824 (mouse), P10107 (mouse), NP_{—}037036 (rat), P14668 (rat), P48037 (rat), Q07936 (rat), P04272 (bovine), P51662 (rabbit), P33477 (rabbit), LURB11 (rabbit), P22464 (drosophila), and P22465 (drosophila). Annexin V (P08133, human) is normally found in high levels in the cytoplasm of a number of cells including placenta, lymphocyte, monocytes, biliary and renal (cortical) tubular epithelium and may be conveniently purified from human placenta (Funakoshi, et al., 1987).

V. Exemplary Infrared Fluorescent Substances

An example of an infrared substance is a quantum dot which may emit at visible light wavelengths, far-red, near-infrared, and infrared wavelengths, and at other wavelengths, typically in response to absorption below their emission wavelength. Quantum dots are a semiconductor nanocrystal with size-dependent optical and electronic properties. In particular, the band gap energy of a quantum dot varies with the diameter of the crystal. Quantum dots (or fluorescent semiconductor nanocrystals) demonstrate quantum confinement effects in their luminescent properties. When quantum dots are illuminated with a primary energy source, a secondary emission of energy occurs of a frequency that corresponds to the band gap of the semiconductor material used in the quantum dot. In quantum confined particles, the band gap is a function of the size of the nanocrystal.

Many semiconductors that are constructed of elements from groups II-VI, III-V and IV of the periodic table have been prepared as quantum sized particles, exhibit quantum confinement effects in their physical properties, and can be used in the composition of the invention. Exemplary materials suitable for use as quantum dots include ZnS, ZnSe, ZnTe, CdS, CdSe, CdTe, GaN, GaP, GaAs, GaSb, InP, InAs, InSb, AlS, AlP, AlAs, AlSb, PbS, PbSe, Ge, and Si and ternary and quaternary mixtures thereof. The quantum dots may further include an overcoating layer of a semiconductor having a greater band gap.

The semiconductor nanocrystals are characterized by their uniform nanometer size. By “nanometer” size, it is meant less than about 150 Angstroms (Å), and preferably in the range of 12-150 Å. The nanocrystals also are substantially monodisperse within the broad nanometer range given above. By monodisperse, as that term is used herein, it is meant a colloidal system in which the suspended particles have substantially identical size and shape. For the purposes of the present invention, monodisperse particles mean that at least 60% of the particles fall within a specified particle size range. Monodisperse particles deviate less than 10% in rms diameter, and preferably less than 5%.

The narrow size distribution of the quantum dots allows the possibility of light emission in narrow spectral widths. Monodisperse quantum dots have been described in detail in Murray et al. (J. Am. Chem. Soc., 115:8706 (1993)); in the thesis of Christopher Murray, “Synthesis and Characterization of II-VI Quantum Dots and Their Assembly into 3-D Quantum Dot Superlattices”, Massachusetts Institute of Technology, September 1995; and in U.S. patent application Ser. No. 08/969,302 entitled “Highly Luminescent Color-selective Materials”.

The fluorescence of semiconductor nanocrystals results from confinement of electronic excitations to the physical dimensions of the nanocrystals. In contrast to the bulk semiconductor material from which these dots are synthesized, these quantum dots have discrete optical transitions, which are tunable with size (U.S. patent application Ser. No. 08/969,302 entitled “Highly Luminescent Color-selective Materials”). Current technology allows good control of their sizes (between 12 to 150 Å; standard deviations approximately 5%), and thus, enables construction of quantum dots that emit light at a desired wavelength throughout the UV-visible-IR spectrum with a quantum yield ranging from 30-50% at room temperature in organic solvents and 10-30% at room temperature in water.

Quantum dots are capable of fluorescence when excited by light. The ability to control the size of quantum dots enables one to construct quantum dots with fluorescent emissions at any wavelength in the UV-visible-IR region. Therefore, the emissions of quantum dots are tunable to any desired spectral wavelength. Furthermore, the emission spectra of monodisperse quantum dots have linewidths as narrow as 25-30 nm. The linewidths are dependent on the size heterogeneity of quantum dots in each preparation.

Appropriate near-infrared fluorescent substances for conjugating to targeting moieties that selectively bind to cells or tissue undergoing cell death, such as annexin V, may have a structure of the formula:

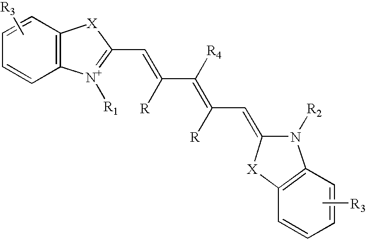

wherein, as valence and stability permit,
X represents C(R)₂, S, Se, O, or NR₅;
R represents H or lower alkyl, or two occurrences of R, taken together, form a ring together with the carbon atoms through which they are connected;
R₁ and R₂ represent, independently, substituted or unsubstituted lower alkyl, lower alkenyl, cycloalkyl, cycloalkylalkyl, aryl, or aralkyl, e.g., optionally substituted by sulfate, phosphate, sulfonate, phosphonate, halogen, hydroxyl, amino, cyano, nitro, carboxylic acid, amide, etc., or a pharmaceutically acceptable salt thereof;
R₃ represents, independently for each occurrence, one or more substituents to the ring to which it is attached, such as a fused ring (e.g., a benzo ring), sulfate, phosphate, sulfonate, phosphonate, halogen, lower alkyl, hydroxyl, amino, cyano, nitro, carboxylic acid, amide, etc., or a pharmaceutically acceptable salt thereof;
R₄ represents H, halogen, or a substituted or unsubstituted ether or thioether of phenol or thiophenol; and
R₅ represents, independently for each occurrence, substituted or unsubstituted lower alkyl, cycloalkyl, cycloalkylalkyl, aryl, or aralkyl, e.g., optionally substituted by sulfate, phosphate, sulfonate, phosphonate, halogen, hydroxyl, amino, cyano, nitro, carboxylic acid, amide, etc., or a pharmaceutically acceptable salt thereof.

Dyes representative of the above formula include indocyanine green, as well as:

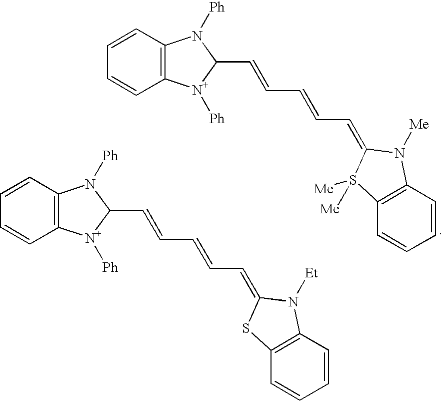

In certain embodiments wherein two occurrences of R taken together form a ring, the ring is six-membered, e.g., the infrared fluorescent dye has a structure of formula: 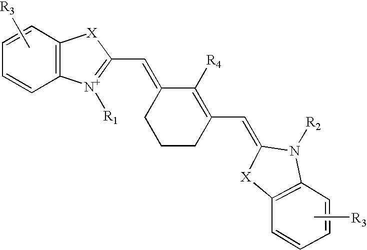
wherein X, R₁, R₂, R₃, R₄, and R₅ represent substituents as described above.

Dyes representative of this formula include IRDye78, IRDye80, IRDye38, IRDye40, IRDye41, IRDye700, IRDye800, Cy7 (AP Biotech), and compounds formed by conjugating a second molecule to any such substance, e.g., a protein or nucleic acid conjugated to IRDye800, IRDye40, or Cy7, etc. The IRDyes are commercially available from Li-Cor Biosciences of Lincoln, Nebr., and each dye has a specified peak absorption wavelength (also referred to herein as the excitation wavelength) and peak emission wavelength that may be used to select suitable optical hardware for use therewith. It will be appreciated that other near-infrared or infrared substances may also be conjugated to a targeting moiety. Several specific dyes suited for specific imaging techniques are now described.

IRDye78-CA is useful for imaging the vasculature of the tissues and organs. The dye in its small molecule form is soluble in blood, and has an in vivo early half-life of several minutes. This permits multiple injections during a single procedure. Indocyanine green has similar characteristics, but is somewhat less soluble in blood and has a shorter half-life.

As another example, IR-786 partitions efficiently into mitochondria and/or endoplasmic reticulum in a concentration-dependent manner, thus permitting blood flow and ischemia visualization in a living heart. The dye has been successfully applied, for example, to image blood flow in the heart of a living laboratory rat after a thoracotomy.

Another example of a near-infrared fluorescent dye is DRAQ5NO, a N-oxide modified anthraquinone. Unlike its non-N modified counterpart, DRAQ5NO has a limited capacity to accumulate in within cells and uptake of DRAQ5NO into a cell is increased when the plasma membrane integrity is compromised, i.e., when the cell undergoes cell death. As such, DRAQ5NO may be used for tracking apoptotic populations in tissues, and thus may enhance a targeting effect. DRAQ5NO is available from Biostatus Limited of Leicestershire, UK.

While a number of suitable dyes have been described, it should be appreciated that such infrared fluorescent substances are examples only, and that more generally, any infrared fluorescent substance may be used with the imaging systems described herein, provided the substance has an emission wavelength that does not interfere with visible light imaging. This includes the near-infrared fluorescent dyes described above, as well as infrared fluorescent substances which may have emission wavelengths above 1000 un, and may be associated with an antibody, antibody fragment, or ligand and imaged in vivo. All such substances are referred to herein as infrared fluorescent substances, and it will be understood that suitable modifications may be made to components of the imaging system for use with any such infrared fluorescent substance.

VI. Imaging Cell Death

The present invention provides, in one aspect, a method of imaging cell death (due, e.g., to apoptosis or necrosis) in a region of a mammalian subject in vivo. In the method, a reagent of the invention, i.e., a targeting moiety, that selectively binds to cells or tissue undergoing cell death, conjugated to an infrared fluorescent substance, preferably a near-infrared fluorescent substance, is administered to the subject. After a period of time in which the reagent can achieve localization in the subject, the area to be imaged is placed within the detection field of a medical imaging device. The area may be maintained in a substantially immobilized condition while emission wavelengths from the reagent are imaged. The image so constructed is then used to provide the attending clinician with a map or a localization of areas of cell death in the mammalian subject, or in the region of the mammalian subject that is being analyzed.

An advantage of the above method is that, by measuring the emission wavelength and forming an image at selected intervals, the method can be used to track changes in the image of emission wavelengths from the subject over time, reflecting changes in the number of cells undergoing cell death. Such an approach may also be used to track changes in the localization of emission wavelengths from the subject over time, reflecting changes in the distribution of cells undergoing cell death.

A. Synthesis of Infrared Fluorescent Substance-Conjugated Targeting Moiety

The invention can be practiced using purified native, recombinant, or synthetically-prepared annexin. The invention is preferably practiced using annexin V, for several reasons: (i) annexin V is one of the most abundant annexins, (ii) it is simple to produce from natural or recombinant sources, and (iii) it has a high affinity for phospholipid membranes (Tait, et al., 1988). Human annexin V has a molecular weight of 36 kd and high affinity (kd=7 nmol/L) for phosphatidylserine (PS). The sequence of human annexin V can be obtained from GenBank under accession numbers U05760-U05770.

Isolated recombinant polypeptides produced above may be purified by standard protein purification procedures, including differential precipitation, molecular sieve chromatography, ion-exchange chromatography, isoelectric focusing, gel electrophoresis and affinity chromatography. Protein preparations can also be concentrated by, for example, filtration (Amicon, Danvers, Mass.).

The invention may be practiced with any one of a variety of infrared fluorescent substances, preferably a near-infrared fluorescent substance, presently available. In selecting a suitable infrared fluorescent substance, the practitioner will typically consider the particular application of the invention, along with factors common to medical imaging in general. Such factors include (i) the excitation wavelength of the infrared fluorescent substance, (ii) energy of a type and in an amount sufficient to cause the substance to fluoresce, (iii) an emission wavelength of the infrared fluorescent substance that does not interfere with visible light imaging, (iv) suitable chemical form and reactivity of the infrared fluorescent substance, and (v) stability or near stability of the infrared fluorescent substance/targeting moiety conjugate.

Forming a reagent of the invention can be accomplished using known techniques. For example, an annexin V/IRDye78 conjugate can be made by reacting annexin V under aqueous conditions to an N-hydroxysuccinimide ester of IRDye78. The unconjugated IRDye78 can be purified from the annexin V/IRDye78 conjugate through gel filtration or dialysis.

A targeting moiety can be linked to an infrared fluorescent substance in a number of ways including by chemical coupling means, or by genetic engineering. Covalent conjugates of a target moiety and an infrared fluorescent substance can be prepared by linking chemical moieties of an infrared fluorescent substance to functional groups on amino acid sidechains or at the N-terminus or at the C-terminus of the target moiety. The subject target moiety may also be chemically modified with other chemical moieties, such as glycosyl groups, lipids, phosphate, acetyl groups and the lice, to facilitate chemical coupling.

To illustrate, there are a large number of chemical cross-linking agents that are known to those skilled in the art. For the present invention, the preferred cross-linking agents are heterobifunctional cross-linkers, which can be used to link a targeting moiety and an infrared fluorescent substance in a stepwise manner. Heterobifunctional cross-linkers provide the ability to design more specific coupling methods for conjugating to proteins, thereby reducing the occurrences of unwanted side reactions such as homo-protein polymers. A wide variety of heterobifunctional cross-linkers are known in the art. These include: succinimidyl 4-(N-maleimidomethyl)cyclohexane-1-carboxylate (SMCC), m-maleimidobenzoyl-N-hydroxysuccinimide ester (MBS); N-succinimidyl (4-iodoacetyl) aminobenzoate (SIAB), succinimidyl 4-(p-maleimidophenyl) butyrate (SMPB), 1-ethyl-3-(3-dimethylaminopropyl) carbodiimide hydrochloride (EDC); 4-succinimidyloxycarbonyl-a-methyl-a-(2-pyridyldithio)-tolune (SMPT), N-succinimidyl 3-(2-pyridyldithio) propionate (SPDP), succinimidyl 6-[3-(2-pyridyldithio) propionate] hexanoate (LC-SPDP). Those cross-linking agents having N-hydroxysuccinimide moieties can be obtained as the N-hydroxysulfosuccinimide analogs, which generally have greater water solubility. In addition, those cross-linking agents having disulfide bridges within the linking chain can be synthesized instead as the alkyl derivatives so as to reduce the amount of linker cleavage in vivo.

In addition to the heterobifunctional cross-linkers, there exist a number of other cross-linking agents including homobifunctional and photoreactive cross-linkers. Disuccinimidyl suberate (DSS), bismaleimidohexane (BMH) and dimethylpimelimidate-2 HCl (DMP) are examples of useful homobifunctional cross-linking agents, and bis-[β-(4-azidosalicylamido)ethyl]disulfide (BASED) and N-succinimidyl-6(4′-azido-2′-nitrophenyl-amino)hexanoate (SANPAH) are examples of useful photoreactive cross-linkers for use in this invention. For a review of protein coupling techniques, see Means et al. (1990) Bioconjugate Chemistry 1:2-12, incorporated by reference herein.

One particularly useful class of heterobifunctional cross-linkers, included above, contain the primary amine reactive group, N-hydroxysuccinimide (NHS), or its water soluble analog N-hydroxysulfosuccinimide (sulfo-NHS). Primary amines (lysine epsilon groups) at alkaline pH's are unprotonated and react by nucleophilic attack on NHS or sulfo-NHS esters. This reaction results in the formation of an amide bond, and release of NHS or sulfo-NHS as a by-product.

Another reactive group useful as part of a heterobifunctional cross-linker is a thiol reactive group. Common thiol reactive groups include maleimides, halogens, and pyridyl disulfides. Maleimides react specifically with free sulfhydryls (cysteine residues) in minutes, under slightly acidic to neutral (pH 6.5-7.5) conditions. Halogens (iodoacetyl functions) react with —SH groups at physiological pH's. Both of these reactive groups result in the formation of stable thioether bonds.

The third component of the heterobifunctional cross-linker is the spacer arm or bridge. The bridge is the structure that connects the two reactive ends. The most apparent attribute of the bridge is its effect on steric hindrance. In some instances, a longer bridge can more easily span the distance necessary to link two complex molecules. For instance, SMPB has a span of 14.5 angstroms.

B. Administration of Infrared Fluorescent Substance-Conjugated Targeting Moiety

A reagent of the invention, an infrared fluorescent substance-conjugated targeting moiety, may be administered using standard protocols for administration of fluorescent compounds. The dosage primarily depends on the amount of targeting moiety injected.

For example, annexin V begins to have pharmacological effects (anti-coagulant effects) at doses greater than about 300 μg protein/kg. Accordingly, the diagnostic methods of the present invention (which seek to avoid pharmacological effects of the conjugated annexin V) are preferably practiced at doses lower than 300 μg protein/kg, typically less than about 50 μg protein/kg, preferably between about 1 and 10 μg protein/kg. Such tracer doses (e.g., 10 μg protein/kg to 50 μg protein/kg) have no reported pharmacologic or toxic side effects in animal or human subjects.

A reagent of the invention is typically suspended in a suitable delivery vehicle, such as sterile saline. The vehicle may also contain stabilizing agents, carriers, excipients, stabilizers, emulsifiers, and the like, as is recognized in the art.

A reagent of the invention can be administered by any of several routes known to be effective for administration of substance-conjugated proteins for nuclear medicine imaging. A preferred method of administration is intravenous (i.v.) injection. It is particularly suitable for imaging of well-vascularized internal organs, such as the heart liver, spleen, etc. Methods for i.v. injection of protein-conjugated compounds are known.

For imaging the brain, the reagent can be administered intrathecally. Intrathecal administration delivers compound directly to the sub-arachnoid space containing cerebral spinal fluid (CSF). Delivery to spinal cord regions can also be accomplished by epidural injection to a region of the spinal cord exterior to the arachnoid membrane.

Other modes of administration include intraperitoneal (e.g., for patients on kidney dialysis), and intrapleural administration. For specific applications, the invention contemplates additional modes of delivery, including intramuscular injection, subcutaneous, intralymphatic, insufflation, and oral, intravesical, intravaginal and/or rectal administration.

Methods for practicing the modes of administration listed above are known in the art.

C. Localization of Infrared Fluorescent Substance-Conjugated Targeting Moiety

After a reagent of the invention is administered, it is allowed to localize to the target tissue or organ. Localization in this context refers to a condition when either an equilibrium or a pseudo-steady state relationship between bound, “localized”, and unbound, “free” reagent, or infrared fluorescent substance-conjugated targeting moiety, within a subject has been achieved. The amount of time required for such localization is typically on the order of minutes to tens of minutes. The localization time also depends on the accessibility of the target tissue to the reagent. This in turn depends on the mode of administration, as is recognized in the art.

Imaging is preferably initiated after most of the reagent has localized to its target(s). One of skill in the art will appreciate, however, that it may be desirable to perform the imaging at times less than or greater than the time to achieve essentially complete localization. For example, in imaging cell death due to blood vessel injury, the accessibility of the target tissue is very high, such that a strong signal can be obtained from the target site in only a few minutes.

A reasonable estimate of the time to achieve localization may be made by one skilled in the art. Furthermore, the state of localization as a function of time may be followed by imaging the signal from the infrared fluorescent substance-conjugated targeting moiety according to the methods of the invention.

VII. Applications

Major uses for a reagent of the invention include the detection of inappropriate apoptosis in disease states where it should not occur, e.g., immune disorders such as lupus, transplant rejection, or in cells subject to severe ischemia; and the detection of insufficient apoptosis when it should occur, e.g., tumors or cells infected with virus.

A reagent of the invention may be employed in a variety of clinical settings in which apoptotic and/or necrotic cell death need to be monitored, such as, without limitation, organ and bone marrow transplant rejection or injury, infectious and non-infectious inflammatory diseases, autoimmune disease, cerebral and myocardial infarction and ischemia, cardiomyopathies, atherosclerotic disease, neural and neuromuscular degenerative diseases, sickle cell disease, β-thalassemia, cancer therapy, AIDS, myelodysplastic syndromes, and toxin-induced liver disease, etc. A reagent of the invention may also be useful as a clinical research reagent to study the normal immune system, embryological development, and immune tolerance and allergy.

A reagent of the invention may be used, for example, to image and quantify apoptotic cell death in normal and malignant tissues undergoing treatment. Monitoring apoptosis with serial imaging studies using an infrared fluorescent substance-conjugated targeting moiety can be used for the rapid testing and development of new drugs and therapies in a variety of diseases. In addition, the compounds may be used to monitor the progress of treatment, monitor the progress of disease, or both. Further, they may be used to aid in early detection of certain diseases.

VIII. Chemical Definitions

‘Acyl’ refers to a group suitable for acylating a nitrogen atom to form an amide or carbamate, a carbon atom to form a ketone, a sulfur atom to form a thioester, or an oxygen atom to form an ester group, e.g., a hydrocarbon attached to a —C(═O)— moiety. Preferred acyl groups include benzoyl, acetyl, tert-butyl acetyl, pivaloyl, and trifluoroacetyl. More preferred acyl groups include acetyl and benzoyl. The most preferred acyl group is acetyl.

The terms ‘amine’ and ‘amino’ are art-recognized and refer to both unsubstituted and substituted amines as well as ammonium salts, e.g., as can be represented by the general formula: 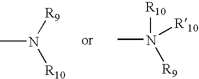
wherein R₉, R₁₀, and R′₁₀ each independently represent hydrogen or a hydrocarbon substituent, or R₉ and R₁₀ taken together with the N atom to which they are attached complete a heterocycle having from 4 to 8 atoms in the ring structure. In preferred embodiments, none of R₉, R₁₀, and R′₁₀ is acyl, e.g., R₉, R₁₀, and R′₁₀ are selected from hydrogen, alkyl, heteroalkyl, aryl, heteroaryl, carbocyclic aliphatic, and heterocyclic aliphatic. The term ‘alkylamine’ as used herein means an amine group, as defined above, having at least one substituted or unsubstituted alkyl attached thereto. Amino groups that are positively charged (e.g., R′₁₀ is present) are referred to as ‘ammonium’ groups. In amino groups other than ammonium groups, the amine is preferably basic, e.g., its conjugate acid has a pKₐ above 7.

The terms ‘amido’ and ‘amide’ are art-recognized as an amino-substituted carbonyl, such as a moiety that can be represented by the general formula: 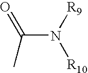
wherein R₉ and R₁₀ are as defined above. In certain embodiments, the amide will include imides.

‘Alkyl’ refers to a saturated or unsaturated hydrocarbon chain having 1 to 18 carbon atoms, preferably 1 to 12, more preferably 1 to 6, more preferably still 1 to 4 carbon atoms. Alkyl chains may be straight (e.g., n-butyl) or branched (e.g., sec-butyl, isobutyl, or t-butyl). Preferred branched alkyls have one or two branches, preferably one branch. Preferred alkyls are saturated. Unsaturated alkyls have one or more double bonds and/or one or more triple bonds. Preferred unsaturated alkyls have one or two double bonds or one triple bond, more preferably one double bond. Alkyl chains may be unsubstituted or substituted with from 1 to 4 substituents. Preferred alkyls are unsubstituted. Preferred substituted alkyls are mono-, di-, or trisubstituted. Preferred alkyl substituents include halo, haloalkyl, hydroxy, aryl (e.g., phenyl, tolyl, alkoxyphenyl, alkyloxycarbonylphenyl, halophenyl), heterocyclyl, and heteroaryl.

The terms ‘alkenyl’ and ‘alkynyl’ refer to unsaturated aliphatic groups analogous in length and possible substitution to the alkyls described above, but that contain at least one double or triple bond, respectively. When not otherwise indicated, the terms alkenyl and alkynyl preferably refer to lower alkenyl and lower alkynyl groups, respectively. When the term alkyl is present in a list with the terms alkenyl and alkynyl, the term alkyl refers to saturated alkyls exclusive of alkenyls and alkynyls.

The terms ‘alkoxyl’ and ‘alkoxy’ as used herein refer to an —O-alkyl group. Representative alkoxyl groups include methoxy, ethoxy, propyloxy, tert-butoxy, and the like. An ‘ether’ is two hydrocarbons covalently linked by an oxygen. Accordingly, the substituent of a hydrocarbon that renders that hydrocarbon an ether can be an alkoxyl, or another moiety such as —O-aryl, —O-heteroaryl, —O-heteroalkyl, —O-aralkyl, —O-heteroaralkyl, —O-carbocylic aliphatic, or —O-heterocyclic aliphatic.

The term ‘aralkyl’, as used herein, refers to an alkyl group substituted with an aryl group.

‘Aryl ring’ refers to an aromatic hydrocarbon ring system. Aromatic rings are monocyclic or fused bicyclic ring systems, such as phenyl, naphthyl, etc. Monocyclic aromatic rings contain from about 5 to about 10 carbon atoms, preferably from 5 to 7 carbon atoms, and most preferably from 5 to 6 carbon atoms in the ring. Bicyclic aromatic rings contain from 8 to 12 carbon atoms, preferably 9 or 10 carbon atoms in the ring. The term ‘aryl’ also includes bicyclic ring systems wherein only one of the rings is aromatic, e.g., the other ring is cycloalkyl, cycloalkenyl, or heterocyclyl. Aromatic rings may be unsubstituted or substituted with from 1 to about 5 substituents on the ring. Preferred aromatic ring substituents include: halo, cyano, lower alkyl, heteroalkyl, haloalkyl, phenyl, phenoxy, or any combination thereof. More preferred substituents include lower alkyl, cyano, halo, and haloalkyl.

‘Cycloalkyl ring’ refers to a saturated or unsaturated hydrocarbon ring. Cycloalkyl rings are not aromatic. Cycloalkyl rings are monocyclic, or are fused, spiro, or bridged bicyclic ring systems. Monocyclic cycloalkyl rings contain from about 4 to about 10 carbon atoms, preferably from 4 to 7 carbon atoms, and most preferably from 5 to 6 carbon atoms in the ring. Bicyclic cycloalkyl rings contain from 8 to 12 carbon atoms, preferably from 9 to 10 carbon atoms in the ring. Cycloalkyl rings may be unsubstituted or substituted with from 1 to 4 substituents on the ring. Preferred cycloalkyl ring substituents include halo, cyano, alkyl, heteroalkyl, haloalkyl, phenyl, phenoxy or any combination thereof. More preferred substituents include halo and haloalkyl. Preferred cycloalkyl rings include cyclopentyl, cyclohexyl, cyclohexenyl, cycloheptyl, and cyclooctyl. More preferred cycloalkyl rings include cyclohexyl, cycloheptyl, and cyclooctyl.

The term ‘carbonyl’ is art-recognized and includes such moieties as can be represented by the general formula: 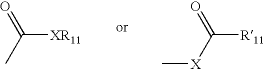
wherein X is a bond or represents an oxygen or a sulfur, and R₁₁ represents a hydrogen, hydrocarbon substituent, or a pharmaceutically acceptable salt, R_{11′} represents a hydrogen or hydrocarbon substituent. Where X is an oxygen and R₁₁ or R_{11′} is not hydrogen, the formula represents an ‘ester’. Where X is an oxygen, and R₁₁ is as defined above, the moiety is referred to herein as a carboxyl group, and particularly when R₁₁ is a hydrogen, the formula represents a ‘carboxylic acid’. Where X is an oxygen, and R_{11′} is hydrogen, the formula represents a ‘formate’. In general, where the oxygen atom of the above formula is replaced by sulfur, the formula represents a ‘thiocarbonyl’ group. Where X is a sulfur and R₁₁ or R_{11′} is not hydrogen, the formula represents a ‘thioester.’ Where X is a sulfur and R₁₁ is hydrogen, the formula represents a ‘thiocarboxylic acid.’ Where X is a sulfur and R_{11′} is hydrogen, the formula represents a ‘thioformate.’ On the other hand, where X is a bond, R₁₁ is not hydrogen, and the carbonyl is bound to a hydrocarbon, the above formula represents a ‘ketone’ group. Where X is a bond, R₁₁ is hydrogen, and the carbonyl is bound to a hydrocarbon, the above formula represents an ‘aldehyde’ or ‘formyl’ group.

‘Ci alkyl’ is an alkyl chain having i member atoms. For example, C4 alkyls contain four carbon member atoms. C4 alkyls containing may be saturated or unsaturated with one or two double bonds (cis or trans) or one triple bond. Preferred C4 alkyls are saturated. Preferred unsaturated C4 alkyl have one double bond. C4 alkyl may be unsubstituted or substituted with one or two substituents. Preferred substituents include lower alkyl, lower heteroalkyl, cyano, halo, and haloalkyl.

‘Halogen’ refers to fluoro, chloro, bromo, or iodo substituents. Preferred halo are fluoro, chloro and bromo; more preferred are chloro and fluoro.

‘Heteroalkyl’ is a saturated or unsaturated chain of carbon atoms and at least one heteroatom, wherein no two heteroatoms are adjacent. Heteroalkyl chains contain from 1 to 18 member atoms (carbon and heteroatoms) in the chain, preferably 1 to 12, more preferably 1 to 6, more preferably still 1 to 4. Heteroalkyl chains may be straight or branched. Preferred branched heteroalkyl have one or two branches, preferably one branch. Preferred heteroalkyl are saturated. Unsaturated heteroalkyl have one or more double bonds and/or one or more triple bonds. Preferred unsaturated heteroalkyl have one or two double bonds or one triple bond, more preferably one double bond. Heteroalkyl chains may be unsubstituted or substituted with from 1 to about 4 substituents unless otherwise specified. Preferred heteroalkyl are unsubstituted. Preferred heteroalkyl substituents include halo, aryl (e.g., phenyl, tolyl, alkoxyphenyl, alkoxycarbonylphenyl, halophenyl), heterocyclyl, heteroaryl. For example, alkyl chains substituted with the following substituents are heteroalkyl: alkoxy (e.g., methoxy, ethoxy, propoxy, butoxy, pentoxy), aryloxy (e.g., phenoxy, chlorophenoxy, tolyloxy, methoxyphenoxy, benzyloxy, alkoxycarbonylphenoxy, acyloxyphenoxy), acyloxy (e.g., propionyloxy, benzoyloxy, acetoxy), carbamoyloxy, carboxy, mercapto, alkylthio, acylthio, arylthio (e.g., phenylthio, chlorophenylthio, alkylphenylthio, alkoxyphenylthio, benzylthio, alkoxycarbonylphenylthio), amino (e.g., amino, mono- and di-C1-C3 alkylamino, methylphenylamino, methylbenzylamino, C1-C3 alkylamido, carbamamido, ureido, guanidino).

‘Heteroatom’ refers to a multivalent non-carbon atom, such as a boron, phosphorous, silicon, nitrogen, sulfur, or oxygen atom, preferably a nitrogen, sulfur, or oxygen atom. Groups containing more than one heteroatom may contain different heteroatoms.

‘Heteroaryl ring’ refers to an aromatic ring system containing carbon and from 1 to about 4 heteroatoms in the ring. Heteroaromatic rings are monocyclic or fused bicyclic ring systems. Monocyclic heteroaromatic rings contain from about 5 to about 10 member atoms (carbon and heteroatoms), preferably from 5 to 7, and most preferably from 5 to 6 in the ring. Bicyclic heteroaromatic rings contain from 8 to 12 member atoms, preferably 9 or 10 member atoms in the ring. The term ‘heteroaryl’ also includes bicyclic ring systems wherein only one of the rings is aromatic, e.g., the other ring is cycloalkyl, cycloalkenyl, or heterocyclyl. Heteroaromatic rings may be unsubstituted or substituted with from 1 to about 4 substituents on the ring. Preferred heteroaromatic ring substituents include halo, cyano, lower alkyl, heteroalkyl, haloalkyl, phenyl, phenoxy or any combination thereof. Preferred heteroaromatic rings include thienyl, thiazolyl, oxazolyl, pyrrolyl, purinyl, pyrimidyl, pyridyl, and furanyl. More preferred heteroaromatic rings include thienyl, furanyl, and pyridyl.

‘Heterocyclic aliphatic ring’ is a non-aromatic saturated or unsaturated ring containing carbon and from 1 to about 4 heteroatoms in the ring, wherein no two heteroatoms are adjacent in the ring and preferably no carbon in the ring attached to a heteroatom also has a hydroxyl, amino, or thiol group attached to it. Heterocyclic aliphatic rings are monocyclic, or are fused or bridged bicyclic ring systems. Monocyclic heterocyclic aliphatic rings contain from about 4 to about 10 member atoms (carbon and heteroatoms), preferably from 4 to 7, and most preferably from 5 to 6 member atoms in the ring. Bicyclic heterocyclic aliphatic rings contain from 8 to 12 member atoms, preferably 9 or 10 member atoms in the ring. Heterocyclic aliphatic rings may be unsubstituted or substituted with from 1 to about 4 substituents on the ring. Preferred heterocyclic aliphatic ring substituents include halo, cyano, lower alkyl, heteroalkyl, haloalkyl, phenyl, phenoxy or any combination thereof. More preferred substituents include halo and haloalkyl. Heterocyclyl groups include, for example, thiophene, thianthrene, furan, pyran, isobenzofuran, chromene, xanthene, phenoxathin, pyrrole, imidazole, pyrazole, isothiazole, isoxazole, pyridine, pyrazine, pyrimidine, pyridazine, indolizine, isoindole, indole, indazole, purine, quinolizine, isoquinoline, hydantoin, oxazoline, imidazolinetrione, triazolinone, quinoline, phthalazine, naphthyridine, quinoxaline, quinazoline, quinoline, pteridine, carbazole, carboline, phenanthridine, acridine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. Preferred heterocyclic aliphatic rings include piperazyl, morpholinyl, tetrahydrofuranyl, tetrahydropyranyl and piperidyl. Heterocycles can also be polycycles.

The term ‘hydroxyl’ means —OH.

‘Lower alkyl’ refers to an alkyl chain comprised of 1 to 4, preferably 1 to 3 carbon member atoms, more preferably 1 or 2 carbon member atoms. Lower alkyls may be saturated or unsaturated. Preferred lower alkyls are saturated. Lower alkyls may be unsubstituted or substituted with one or about two substituents. Preferred substituents on lower alkyl include cyano, halo, trifluoromethyl, amino, and hydroxyl. Throughout the application, preferred alkyl groups are lower alkyls. In preferred embodiments, a substituent designated herein as alkyl is a lower alkyl. Likewise, ‘lower alkenyl’ and ‘lower alkynyl’ have similar chain lengths.

‘Lower heteroalkyl’ refers to a heteroalkyl chain comprised of 1 to 4, preferably 1 to 3 member atoms, more preferably 1 to 2 member atoms. Lower heteroalkyl contain one or two non-adjacent heteroatom member atoms. Preferred lower heteroalkyl contain one heteroatom member atom. Lower heteroalkyl may be saturated or unsaturated. Preferred lower heteroalkyl are saturated. Lower heteroalkyl may be unsubstituted or substituted with one or about two substituents. Preferred substituents on lower heteroalkyl include cyano, halo, trifluoromethyl, and hydroxyl.

‘Mi heteroalkyl’ is a heteroalkyl chain having i member atoms. For example, M4 heteroalkyls contain one or two non-adjacent heteroatom member atoms. M4 heteroalkyls containing 1 heteroatom member atom may be saturated or unsaturated with one double bond (cis or trans) or one triple bond. Preferred M4 heteroalkyl containing 2 heteroatom member atoms are saturated. Preferred unsaturated M4 heteroalkyl have one double bond. M4 heteroalkyl may be unsubstituted or substituted with one or two substituents. Preferred substituents include lower alkyl, lower heteroalkyl, cyano, halo, and haloalkyl.

‘Member atom’ refers to a polyvalent atom (e.g., C, O, N, or S atom) in a chain or ring system that constitutes a part of the chain or ring. For example, in cresol, six carbon atoms are member atoms of the ring and the oxygen atom and the carbon atom of the methyl substituent are not member atoms of the ring.

As used herein, the term ‘nitro’ means —NO₂.

‘Pharmaceutically acceptable salt’ refers to a cationic salt formed at any acidic (e.g., hydroxamic or carboxylic acid) group, or an anionic salt formed at any basic (e.g., amino or guanidino) group. Such salts are well known in the art. See e.g., World Patent Publication 87/05297, Johnston et al., published Sep. 11, 1987, incorporated herein by reference. Such salts are made by methods known to one of ordinary skill in the art. It is recognized that the skilled artisan may prefer one salt over another for improved solubility, stability, formulation ease, price and the like. Determination and optimization of such salts is within the purview of the skilled artisan's practice. Preferred cations include the alkali metals (such as sodium and potassium), and alkaline earth metals (such as magnesium and calcium) and organic cations, such as trimethylammonium, tetrabutylammonium, etc. Preferred anions include halides (such as chloride), sulfonates, carboxylates, phosphates, and the like. Clearly contemplated in such salts are addition salts that may provide an optical center where once there was none. For example, a chiral tartrate salt may be prepared from the compounds of the invention. This definition includes such chiral salts.

‘Phenyl’ is a six-membered monocyclic aromatic ring that may or may not be substituted with from 1 to 5 substituents. The substituents may be located at the ortho, meta or para position on the phenyl ring, or any combination thereof. Preferred phenyl substituents include: halo, cyano, lower alkyl, heteroalkyl, haloalkyl, phenyl, phenoxy or any combination thereof. More preferred substituents on the phenyl ring include halo and haloalkyl. The most preferred substituent is halo.

The terms ‘polycyclyl’ and ‘polycyclic group’ refer to two or more rings (e.g., cycloalkyls, cycloalkenyls, heteroaryls, aryls and/or heterocyclyls) in which two or more member atoms of one ring are member atoms of a second ring. Rings that are joined through non-adjacent atoms are termed ‘bridged’ rings, and rings that are joined through adjacent atoms are ‘fused rings’.

The term ‘sulfate’ is art-recognized and includes a moiety that can be represented by the general formula: 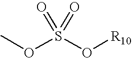
in which R₁₀ is as defined above.

A ‘substitution’ or ‘substituent’ on a small organic molecule generally refers to a position on a multivalent atom bound to a moiety other than hydrogen, e.g., a position on a chain or ring exclusive of the member atoms of the chain or ring. Such moieties include those defined herein and others as are known in the art, for example, halogen, alkyl, alkenyl, alkynyl, azide, haloalkyl, hydroxyl, carbonyl (such as carboxyl, alkoxycarbonyl, formyl, ketone, or acyl), thiocarbonyl (such as thioester, thioacetate, or thioformate), alkoxyl, phosphoryl, phosphonate, phosphinate, amine, amide, amidine, imine, cyano, nitro, azido, sulfhydryl, alkylthio, sulfate, sulfonate, sulfamoyl, sulfonamido, sulfonyl, silyl, ether, cycloalkyl, heterocyclyl, heteroalkyl, heteroalkenyl, and heteroalkynyl, heteroaralkyl, aralkyl, aryl or heteroaryl. It will be understood by those skilled in the art that certain substituents, such as aryl, heteroaryl, polycyclyl, alkoxy, alkylamino, alkyl, cycloalkyl, heterocyclyl, alkenyl, alkynyl, heteroalkyl, heteroalkenyl, and heteroalkynyl, can themselves be substituted, if appropriate. This invention is not intended to be limited in any manner by the permissible substituents of organic compounds. It will be understood that ‘substitution’ or ‘substituted with’ includes the implicit proviso that such substitution is in accordance with permitted valence of the substituted atom and the substituent, and that the substitution results in a stable compound, e.g., which does not spontaneously undergo transformation such as by rearrangement, cyclization, elimination, hydrolysis, etc.

As used herein, the definition of each expression, e.g., alkyl, m, n, etc., when it occurs more than once in any structure, is intended to be independent of its definition elsewhere in the same structure.

The abbreviations Me, Et, Ph, Tf, Nf, Ts, and Ms represent methyl, ethyl, phenyl, trifluoromethanesulfonyl, nonafluorobutanesulfonyl, p-toluenesulfonyl, and methanesulfonyl, respectively. A more comprehensive list of the abbreviations utilized by organic chemists of ordinary skill in the art appears in the first issue of each volume of the Journal of Organic Chemistry; this list is typically presented in a table entitled Standard List of Abbreviations. The abbreviations contained in said list, and all abbreviations utilized by organic chemists of ordinary skill in the art are hereby incorporated by reference.

For purposes of this invention, the chemical elements are identified in accordance with the Periodic Table of the Elements, CAS version, Handbook of Chemistry and Physics, 67th Ed., 1986-87, inside cover. Also for purposes of this invention, the term ‘hydrocarbon’ is contemplated to include all permissible compounds or moieties having at least one carbon-hydrogen bond. In a broad aspect, the permissible hydrocarbons include acyclic and cyclic, branched and unbranched, carbocyclic and heterocyclic, aromatic and nonaromatic organic compounds which can be substituted or unsubstituted.

### DETAILED DESCRIPTION OF CERTAIN EMBODIMENTS OF THE INVENTION

To provide an overall understanding of the invention, certain illustrative embodiments will now be described, including a system for generating superimposed circulatory and tissue images in video format. However, it will be understood that the methods and systems described herein can be suitably adapted to other medical imaging applications where visible light tissue images may be usefully displayed with diagnostic image information obtained from outside the visible light range and superimposed onto the visible light image. More generally, the methods and reagents described herein may be adapted to any imaging application where a visible light image may be usefully displayed with a superimposed image captured from areas within the visible light image that are functionally marked to emit photons outside the visible light range by a substance or other material. For example, the reagents and methods are applicable to a wide range of diagnostic or surgical applications where a target pathology, tissue type, or cell may be labeled with an infrared fluorescent dye or other infrared fluorescent substance, such as a near-infrared fluorescent substance conjugated to a targeting moiety that selectively binds to cells or tissue undergoing cell death. These and other applications of the systems described herein are intended to fall within the scope of the invention.

In one aspect of the invention, the reagent of the invention comprises a targeting moiety that selectively binds to or localizes to the site of cells or tissue undergoing cell death, the protein being covalently conjugated to an infrared fluorescent substance, preferably a near-infrared fluorescent substance.

In certain embodiments, the infrared fluorescent substance is a near-infrared dye having a structure of the formula:

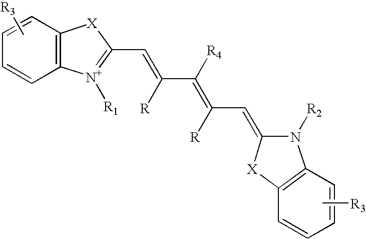

wherein, as valence and stability permit,
X represents C(R)₂, S, Se, O, or NR₅;
R represents H or lower alkyl, or two occurrences of R, taken together, form a ring together with the carbon atoms through which they are connected;
R₁ and R₂ represent, independently, substituted or unsubstituted lower alkyl, lower alkenyl, cycloalkyl, cycloalkylalkyl, aryl, or aralkyl, e.g., optionally substituted by sulfate, phosphate, sulfonate, phosphonate, halogen, hydroxyl, amino, cyano, nitro, carboxylic acid, amide, etc., or a pharmaceutically acceptable salt thereof;
R₃ represents, independently for each occurrence, one or more substituents to the ring to which it is attached, such as a fused ring (e.g., a benzo ring), sulfate, phosphate, sulfonate, phosphonate, halogen, lower alkyl, hydroxyl, amino, cyano, nitro, carboxylic acid, amide, etc., or a pharmaceutically acceptable salt thereof;
R₄ represents H, halogen, or a substituted or unsubstituted ether or thioether of phenol or thiophenol; and
R₅ represents, independently for each occurrence, substituted or unsubstituted lower alkyl, cycloalkyl, cycloalkylalkyl, aryl, or aralkyl, e.g., optionally substituted by sulfate, phosphate, sulfonate, phosphonate, halogen, hydroxyl, amino, cyano, nitro, carboxylic acid, amide, etc., or a pharmaceutically acceptable salt thereof.

In some embodiments, two occurrences of R taken together form a six-membered ring.

Another aspect of the invention provides a method of imaging cell death comprising

(a) contacting a sample of cells with a reagent as described herein,

(b) positioning the sample adjacent to an electronic imaging device, and

(c) constructing an image of emission wavelength,

wherein said image is a representation of cell death in said sample.

In certain embodiments, a pharmaceutical preparation comprises a reagent of the invention and a pharmaceutically acceptable excipient. In some embodiments, a composition comprises a reagent as described herein.

In a preferred embodiment, the targeting moiety is a protein or a fragment thereof. In certain embodiments, the targeting moiety is a protein, or a fragment thereof, having an amino acid sequence at least 60%, or more preferably at least 70%, 80%, 90%, 95%, 98%, homologous to the amino acid sequence of an annexin protein. In a preferred embodiment, the protein, or a fragment thereof, having an amino acid sequence at least 60%, or more preferably at least 70%, 80%, 90%, 95%, 98%, homologous to the amino acid sequence of an annexin protein has at least one biological activity of an annexin protein, such as having a high affinity for anionic phospholipids surfaces or binding to phosphatidylserine.

In certain embodiments, the infrared fluorescent substance is iodocyanine green, IRDye78, IRDye80, IRDye38, IRDye40, IRDye41, IRDye700, IRDye800, Cy7, IR-786, DRAQ5NO, or analogs thereof. In some embodiments, annexin V is conjugated to the N-hydroxysuccinimide ester of IRDye78. In a preferred embodiment, the targeting moiety is annexin V. In a preferred embodiment, the targeting moiety is annexin V and the infrared fluorescent substance is IRDye78.

In a preferred embodiment, the infrared fluorescent substance is a quantum dot or an analog thereof. In a preferred embodiment, the targeting moiety is annexin V and the infrared fluorescent substance is a quantum dot or an analog thereof. In certain embodiments, the targeting moiety-conjugated infrared fluorescent substance is purified from the unconjugated infrared fluorescent substance through gel filtration or dialysis.

In some embodiments, the reagent of the invention may be soluble in blood. In some embodiments, the reagent of the invention may be used to image organ and bone marrow transplant rejection or injury, infectious and non-infectious inflammatory diseases, autoimmune disease, cerebral and myocardial infarction and ischemia, cardiomyopathies, atherosclerotic disease, neural and neuromuscular degenerative diseases, sickle cell disease, β-thalassemia, cancer therapy, AIDS, myelodysplastic syndromes, such as aplastic anemia, toxin-induced liver disease, traumatic injury, bacterial infection, or acute hypoxia. In certain embodiments, the targeting moiety-conjugated substance may be used to image ischemic injury. The ischemic injury may be myocardial infarction, reperfusion injury or stroke.

In another aspect of the invention, the reagent of the invention may be used in a system comprising:

a visible light source providing light over a range of wavelengths that includes one or more wavelengths of visible light;

an excitation light source providing light at one or more wavelengths outside the range of wavelengths of the visible light source, the one or more wavelengths selected to excite a reagent of the invention, the reagent which emits one or more photons at an emission wavelength;

an electronic imaging device;

an optical guide having a first end with a lens that captures an image of a subject and a second end that couples the image to the electronic imaging device; and

a filter for coupling the visible light source and the excitation light source into the optical guide, the filter reflecting some of the light provided by the visible light source and some of the light from the excitation light source toward the subject, the filter further transmitting some visible light from the subject captured by the lens toward the electronic imaging device, and the filter further transmitting the emission wavelength from the subject captured by the lens toward the electronic imaging device.

In another aspect of the invention, the reagent of the invention may be used in a system comprising:

a visible light source illuminating a subject, the visible light source providing a range of wavelengths including one or more wavelengths of visible light;

an excitation light source illuminating the subject, the excitation light source providing an excitation wavelength that is not one of the one or more wavelengths of visible light;

a composition comprising a reagent of the invention introduced into a circulatory system of the subject, the composition being soluble in blood carried by the circulatory system and the composition emitting photons at an emission wavelength in response to the excitation wavelength;

an electronic imaging device that captures an image of a field of view that includes some portion of the subject and the circulatory system of the subject, the image including a first image obtained from the one or more wavelengths of visible light and a second image obtained from the emission wavelength; and

a display that renders the first image and the second image, the second image being displayed at a visible light wavelength.

In yet another aspect of the invention, the reagent of the invention may be used in a system comprising:

an operating area closed to ambient light, the operating area including a surgical field where a surgical procedure may be performed on a subject;

a visible light source illuminating the surgical field, the visible light source providing a range of wavelengths including one or more wavelengths of visible light;

an excitation light source illuminating the surgical field, the excitation light source including at least one wavelength outside the range of wavelengths of visible light;

a composition comprising a reagent of the invention suitable for in vivo use, the reagent fluorescing at an emission wavelength in response to the at least one wavelength of the excitation light source, the composition being introduced into the surgical field;

an electronic imaging device that captures a visible light image of the surgical field and an emission wavelength image of the surgical field; and

a display that renders the visible light image and the emission wavelength image of the surgical field, the emission wavelength image being displayed at a visible light wavelength.

In still another aspect of the invention, the reagent of the invention may be used in a system comprising:

a visible light source that illuminates a subject, the visible light source providing a range of wavelengths including one or more wavelengths of visible light;

an excitation light source that illuminates the subject at the same time that the visible light source illuminates the subject, the excitation light source providing an excitation wavelength that is not one of the one or more wavelengths of visible light;

a composition comprising a reagent of the invention introduced into a circulatory system of the subject, the reagent being soluble in blood carried by the circulatory system and the reagent emitting photons at an emission wavelength in response to the excitation wavelength; and

an electronic imaging device that captures an image of a field of view that includes some portion of the subject and the circulatory system of the subject, the image including a first image obtained from the one or more wavelengths of visible light and a second image concurrently obtained from the emission wavelength.

In another aspect of the invention, the reagent of the invention may be used in a method of imaging cell death within a region in vivo, comprising

(a) administering to a subject the composition comprising an infrared fluorescent substance conjugated to a targeting moiety binding to the membrane of dead cells,

(b) positioning the subject within an electronic imaging device, and

(c) constructing an image of emission wavelength,

wherein said image is a representation of cell death within a region of said subject.

In certain embodiments, the electronic imaging device captures an image of a field of view that includes some portion of the subject and the circulatory system of the subject, the image including a first image obtained from the one or more wavelengths of visible light and a second image obtained from the emission wavelength. In some embodiments, the electronic imaging device captures a visible light image of the surgical field and an emission wavelength image of the surgical field. In other embodiments, the electronic imaging device captures an image of a field of view that includes some portion of the subject and the circulatory system of the subject, the image including a first image obtained from the one or more wavelengths of visible light and a second image concurrently obtained from the emission wavelength.

In some embodiments, the reagent of the invention or a preparation of the reagent is administered intravenously, intraperitoneally, intrathecally, intrapleurally, intralymphatically, intravaginally, intravesically, intrarectally, or intramuscularly. In other embodiments, the reagent of the invention is topically applied.

In certain embodiments, the fluorescent substance will have an emission wavelength in a range from about 680 nm to about 100,000 nm. In preferred embodiments, the reagent according will have an emission wavelength in a range from about 680 nm to about 20,000 nm, even more preferably in a range from about 680 nm to about 15,000 nm.

In certain embodiments, the reagent, or preparation, of the invention is administered in a dose of less than 300 μg protein/kg. In preferred embodiments, the reagent is administered in a dose of less than 50 μg protein/kg or, even more preferably, in a dose of less than 10 μg protein/kg.

In certain embodiments, the infrared fluorescent substance is a near-infrared fluorescent dye having a structure of the formula:

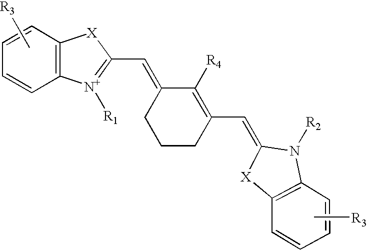

wherein, as valence and stability permit,
X represents C(R)₂, S, Se, O, or NR₅;
R₁ and R₂ represent, independently, substituted or unsubstituted lower alkyl, lower alkenyl, cycloalkyl, cycloalkylalkyl, aryl, or aralkyl, e.g., optionally substituted by sulfate, phosphate, sulfonate, phosphonate, halogen, hydroxyl, amino, cyano, nitro, carboxylic acid, amide, etc., or a pharmaceutically acceptable salt thereof;
R₃ represents, independently for each occurrence, one or more substituents to the ring to which it is attached, such as a fused ring (e.g., a benzo ring), sulfate, phosphate, sulfonate, phosphonate, halogen, lower alkyl, hydroxyl, amino, cyano, nitro, carboxylic acid, amide, etc., or a pharmaceutically acceptable salt thereof;
R₄ represents H, halogen, or a substituted or unsubstituted ether or thioether of phenol or thiophenol; and
R₅ represents, independently for each occurrence, substituted or unsubstituted lower alkyl, cycloalkyl, cycloalkylalkyl, aryl, or aralkyl, e.g., optionally substituted by sulfate, phosphate, sulfonate, phosphonate, halogen, hydroxyl, amino, cyano, nitro, carboxylic acid, amide, etc., or a pharmaceutically acceptable salt thereof.

In certain embodiments, the near-infrared fluorescent dye having a structure of the above formula is selected from IRDye78, IRDye80, IRDye38, IRDye40, IRDye41, IRDye700, IRDye800, Cy7, and compounds formed by conjugating a second molecule to any of IRDye78, IRDye80, IRDye38, IRDye40, IRDye41, IRDye700, IRDye800, and Cy7.

Another aspect of the invention provides a method for detecting cell death comprising

(a) treating the sample with a reagent as described herein,

(b) irradiating the sample with a light source,

(c) detecting an emission wavelength of the substance.

Contemplated equivalents of the compounds described above include compounds which otherwise correspond thereto, and which have the same useful properties thereof, wherein one or more simple variations of substituents are made which do not adversely affect the efficacy of the compound. In general, the compounds of the present invention may be prepared by the methods illustrated in the general reaction schemes as, for example, described below, or by modifications thereof, using readily available starting materials, reagents and conventional synthesis procedures. In these reactions, it is also possible to make use of variants that are in themselves known, but are not mentioned here.

### DETAILED DESCRIPTION OF THE FIGURES

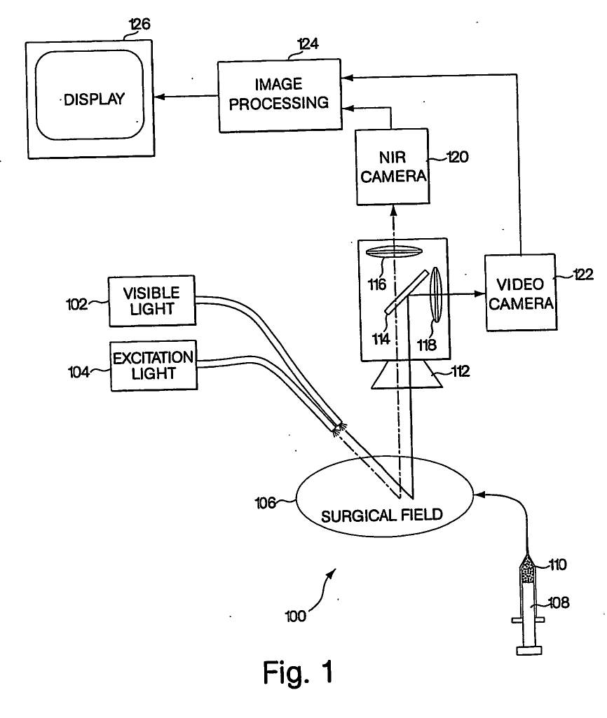

FIG. 1 shows an embodiment of an imaging system for use during open surgery. The imaging system 100 may include a visible light source 102, and excitation light source 104, a surgical field 106, an targeting moiety-conjugated infrared fluorescent substance source 108 containing a targeting moiety-conjugated infrared fluorescent substance 110 (reagent of the invention), a lens 112, a first filter 114, a second filter 116, a third filter 118, an infrared camera 120, a video camera 122, an image processing unit 124, and a display 126. In general, the visible light source 102 and the excitation light source 104 illuminate the surgical field 106. The targeting moiety-conjugated infrared fluorescent substance 110 may be introduced from the targeting moiety-conjugated infrared fluorescent substance source 108, such as through injection into the bloodstream of a subject. An image from the surgical field 106 is then captured by two cameras, the video camera 122 capturing a conventional, visible light image of the surgical field 106 and the infrared camera 120 capturing a diagnostic image based upon the distribution of the targeting moiety-conjugated infrared fluorescent substance 110 in the surgical field 106. These images may be combined by the image processing unit 124 and presented on a display 126 where they may be used, for example, by a surgeon conducting a surgical procedure. Each aspect of the system 100 is now described in more detail.

The imaging system 100 may be surrounded by an operating area (not shown) closed to ambient light. As will become clear from the following, many visible light sources such as incandescent lamps, halogen lamps, or daylight may include a broad spectrum of electromagnetic radiation that extends beyond the range of visible light detected by the human eye and into wavelengths used in the present system as a separate optical channel for generating diagnostic images. In order to effectively detect emission in these super-visible light wavelengths, it is preferred to enclose the surgical field 106, light sources 102, 104, and cameras 120, 122 in an area that is not exposed to broadband light sources. This may be achieved by using an operating room closed to external light sources, or by using a hood or other enclosure or covering for the surgical field 106 that prevents invasion by unwanted spectrum. The visible light source 102 may then serve as a light source for the visible light camera 122, and also for provide conventional lighting within the visible light spectrum. As used herein, the term “operating area” is intended specifically to refer to an open surgical site that is closed to ambient light. Endoscopic or laparoscopic applications, as described below, are confined to surgical procedures within a closed body cavity, and do not include an operating area as that term is intended herein.

The visible light source 102 may be, for example, an infrared depleted white light source. This may be a one-hundred fifty Watt halogen lamp with one or more filters to deplete wavelengths greater than 700 nanometers (“nm”). Generally, any light source constrained to wavelengths between 400 nm and 700 nm may operate as the visible light source 102. In certain applications, the excitation light source 104 and resulting emission from the targeting moiety-conjugated infrared fluorescent substance 110 may have wavelengths near or below 700 nm. These near-red substances may be used with the system, however, this requires a visible light source 102 that excludes a portion of the visible light spectrum in which the substance operates, i.e., a far-red depleted white light source. Similarly, applications using quantum dots as a fluorescent substance may have absorption or emission wavelengths anywhere in the visible light spectrum, and a suitable visible light source should be depleted at the wavelength(s) of interest. As such, the visible light source 102 should more generally be understood to be a source of light that includes some, but not necessarily all, of the wavelengths of visible light.

It should also be understood that, in a far-red imaging system or infrared imaging system such as those noted above, the infrared camera 120 described in the example embodiment will instead be a camera sensitive to the emission wavelength of the targeting moiety-conjugated infrared fluorescent substance 110 or other infrared fluorescent substance, and that other modifications to light sources, filters and other optics will be appropriate. Similar modifications may be made to isolate a band of wavelengths for targeting moiety-conjugated infrared fluorescent substance excitation and emission anywhere within or outside the visible light range, provided that suitable optics, cameras, and targeting moiety-conjugated infrared fluorescent substances are available. Other infrared fluorescent substances may also be used. Suitable adjustments will be made to the excitation light source 104 and the emission camera, the infrared camera 120 in the example embodiment, for such applications. Cameras sensitive to far-red, near-infrared, and infrared wavelengths are commercially available.

The excitation light source 104 provides light at a wavelength that excites the targeting moiety-conjugated infrared fluorescent substance 110. This may be, for example, a laser diode such as a 771 nm, 250 mW laser diode system, which may be obtained from Laser Components of Santa Rosa, Calif. Other single wavelength, narrowband, or broadband light sources may be used, provided they do not interfere with the visible light image captured by the video camera 122 or the emission wavelength of the targeting moiety-conjugated infrared fluorescent substance 110. The infrared band is generally understood to include wavelengths between 700 nm and 1000 nm, and is a useful wavelength range for a number of readily available excitation light sources 104 and targeting moiety-conjugated infrared fluorescent substances 110 that may be used with the systems described herein. Suitable optical coupling and lenses may be provided to direct each of the visible light source 102 and the excitation light source 104 at an area of interest within the surgical field 106.

The surgical field 106 may be any area of a subject or patient that is open for a surgical procedure. This may be, for example, an open chest during a procedure such as a revascularization or cardiac gene therapy, where visualization of the circulatory system may improve identification of areas at risk for myocardial infarction. Blood flow visualization may permit an assessment of coronary arteries during a coronary artery bypass graft, or an assessment of blood flow and viability during introduction of genes for endothelial growth factor or fibroblast growth factor to induce neovascularization within ischemic regions of the heart. More generally, the surgical field 106 may include any areas of a patient's body, such as a region of the body that includes a tumor that is to be surgically removed, and that is amenable to visualization with fluorescent substances, such as through the use of labeled antibodies.

The targeting moiety-conjugated infrared fluorescent substance source 108 may be any instrument used for injection or other introduction of the targeting moiety-conjugated infrared fluorescent substance 110 into a subject, such as a hypodermic needle or angiocath. Where, for example, the targeting moiety-conjugated infrared fluorescent substance 110 is highly soluble in blood, the targeting moiety-conjugated infrared fluorescent substance source 108 may be administered anywhere on the subject, and need not be near the surgical field 106. In certain embodiments, the targeting moiety-conjugated infrared fluorescent substance source 108 may not use injection. For example, the targeting moiety-conjugated infrared fluorescent substance source 108 may spray or otherwise apply the targeting moiety-conjugated infrared fluorescent substance 110 to an area of interest. Depending upon the type of substance and the imaging technique, the targeting moiety-conjugated infrared fluorescent substance 110 may be delivered in a discrete dose, or may be continuously or intermittently applied and re-applied by the targeting moiety-conjugated infrared fluorescent substance source 108.

The targeting moiety-conjugated infrared fluorescent substance 110 may be any substance suitable for use in vivo and having excitation and emission wavelengths suitable for other components of the system 100. Typically, the targeting moiety-conjugated infrared fluorescent substance 110 will be diluted to 25-50 μM for intravenous injection, such as with phosphate buffered saline, which may be supplemented with Cremophor EL (Sigma) and/or absolute ethanol. A number of suitable infrared substances are described above.

The lens 112 may be any lens suitable for receiving light from the surgical field 106 and focusing the light for image capture by the infrared camera 120 and the video camera 122. The lens 112 may include one or more optical coatings suitable for the wavelengths to be imaged, and may provide for manual, electronically-assisted manual, or automatic control of zoom and focus.

The first filter 114 may be positioned in the image path from the lens 112 such that a visible light image having one or more visible light wavelengths is directed toward the video camera 122, either by reflection or transmittance. An emission image from the excited targeting moiety-conjugated infrared fluorescent substance 110 passes through the lens 112 and is directed toward the near infrared camera 120, again either through reflection or transmittance. A number of arrangements of the cameras 120, 122 and the first filter 114 are possible, and may involving reflecting or transmitting either the visible light image or the emission wavelength image.

In one embodiment, IRDye78-CA (carboxylic acid) having a peak absorption near 771 nm and a peak emission near 806 nm, is used with the system 100. In this embodiment, the first filter 114 may be a 785 nm dichroic mirror that transmits infrared light and reflects visible light. The first filter 114 may be positioned within an image path from the lens 112 such that a visible light image of the surgical field 106 is reflected toward the video camera 122 through the third filter 118. The third filter 118 may be, for example, a 400 nm-700 nm visible light filter. At the same time, the first filter 114 is positioned with the image path from the lens 112 such that an infrared image (i.e., the excitation wavelength image) is transmitted toward the infrared camera 120 through the second filter 116. The second filter 116 may be an 810 nm+/−20 nm near-infrared emission filter. The filters may be standard or custom-ordered optical components, which are commercially available from optical component suppliers. Other arrangements of filters and other optical components may be used with the system 100 described herein.

The infrared camera 120 may be any still or moving image camera suitable for capturing images at the emission wavelength of the excited targeting moiety-conjugated infrared fluorescent substance 110. The infrared camera may be, for example, an Orca-ER infrared camera with settings of gain 7, 2×2 binning, 640×480 pixel field of view, and an exposure time of 20 msec and an effective frame rate of fifteen frames per second. The Orca-ER is commercially available from Hamamatsu Photonic Systems of Bridgewater, N.J. It will be understood that the infrared camera 120 of FIG. 1 is only an example. An infrared camera, a farred camera, or some other camera or video device may be used to capture an emission wavelength image, with the camera and any associated filters selected according to the wavelength of a corresponding infrared fluorescent substance used with the imaging system. As used herein, the term “emission wavelength camera” is intended to refer to any such camera that may be used with the systems described herein.

The video camera 122 may be any video camera suitable for capturing images of the surgical field 106 in the visible light spectrum. In one embodiment, the video camera 122 is a color video camera model HV-D27, commercially available from Hitachi of Tarrytown, N.Y. The video camera 122 may capture red-green-blue (RGB) images at thirty frames per second at a resolution of 640×480 pixels. More generally, the infrared camera 120 and the video camera 122 may be any device capable of photonic detection and conversion to electronic images, including linear photodiode arrays, charge coupled device arrays, scanning photomultiplier tubes, and so forth.

The display 126 may be a television, high-definition television, computer monitor, or other display configured to receive and render signals from the image processing unit 124. The surgical field 106 may also be a neurosurgical site, with a surgical microscope used to view the surgical field 106. In this embodiment, the display 126 may be a monocular or binocular eyepiece of the surgical microscope, with the infrared image superimposed on the visible light image in the eyepiece. In another embodiment, the eyepiece may use direct optical coupling of the surgical field 106 to the eyepiece for conventional microscopic viewing, with the infrared image projected onto the eyepiece using, for example, heads-up display technology.

The image processing unit 124 may include any software and/or hardware suitable for receiving images from the cameras 120, 122, processing the images as desired, and transmitting the images to the display 126. In one embodiment, the image processing unit 124 is realized in software on a Macintosh computer equipped with a Digi-16 Snapper frame grabber for the Orca-ER, commercially available from DataCell of North Billerica, Mass., and equipped with a CG-7 frame grabber for the HV-D27, commercially available from Scion of Frederick Md., and using IPLab software, commercially available from Sanalytics of Fairfax, Va. While a Macintosh may be used in one embodiment, any general purpose computer may be programmed to perform the image processing functions described herein, including an Intel processor-based computer, or a computer using hardware from Sun Microsystems, Silicon Graphics, or any other microprocessor manufacturer.

Generally, the image processing unit 124 should be capable of digital filtering, gain adjustment, color balancing, and any other conventional image processing functions. The image from the infrared camera 120 is also typically shifted into the visible light range for display at some prominent wavelength, e.g., a color distinct from the visible light colors of the surgical field 106, so that a superimposed image will clearly depict the annexin V-conjugated infrared fluorescent substance. The image processing unit 124 may also perform image processing to combine the image from the infrared camera 120 and the video camera 122. Where the images are displayed side-by-side, this may simply entail rendering the images in suitable locations on a computer screen. Where the images are superimposed, a frame rate adjustment may be required. That is, if the video camera 122 is capturing images at the conventional rate of thirty frames per second and the infrared camera 120 is taking still pictures with an effective frame rate of fifteen frames per second, some additional processing may be required to render the superimposed images concurrently. This may entail either reducing the frame rate of the video camera 122 to the frame rate of the infrared camera 120 either by using every other frame of video data or averaging or otherwise interpolating video data to a slower frame rate. This may instead entail increasing the frame rate of the infrared image data, either by holding each frame of infrared data over successive frames of video data or extrapolating infrared data, such as by warping the infrared image according to changes in the video image or employing other known image processing techniques.

Generally, any combination of software or hardware may be used in the image processing unit 124. The functions of the image processing unit 124 may be realized, for example, in one or more microprocessors, microcontrollers, embedded microcontrollers, programmable digital signal processors or other programmable device, along with internal and/or external memory such as read-only memory, programmable read-only memory, electronically erasable programmable read-only memory, random access memory, dynamic random access memory, double data rate random access memory, Rambus direct random access memory, flash memory, or any other volatile or non-volatile memory for storing program instructions, program data, and program output or other intermediate or final results. The functions may also, or instead, include one or more application specific integrated circuits, programmable gate arrays, programmable array logic devices, or any other device or devices that may be configured to process electronic signals. Any combination of the above circuits and components, whether packaged discretely, as a chip, as a chipset, or as a die, may be suitably adapted to use with the systems described herein.

It will further be appreciated that each function of the image processing unit 124 may be realized as computer executable code created using a structured programming language such as C, an object-oriented programming language such as C++ or Java, or any other high-level or low-level programming language that may be compiled or interpreted to run on one of the above devices, as well as heterogeneous combinations of processors, processor architectures, or combinations of different hardware and software. The image processing unit 124 may be deployed using software technologies or development environments including a mix of software languages, such as Java, C++, Oracle databases, SQL, and so forth. It will be further appreciated that the functions of the image processing unit 124 may be realized in hardware, software, or some combination of these.

In one embodiment, the visible light source 102 is an infrared depleted visible light source, the excitation light source 104 is a 771 nm, 250 mW laser diode, the targeting moiety-conjugated infrared fluorescent substance 110 is targeting moiety-conjugated to indocyanine green or IRDye78-CA, the first filter 114 is a 785 nm dichroic mirror configured to transmit infrared light and reflect visible light, the second filter 116 is an 810 nm+/−20 nm infrared emission filter, and the third filter 118 is a 400 nm to 700 nm filter. The image processing unit 124 is a computer with software for image capture from the infrared camera 120 and the video camera 122, for making suitable color adjustment to the images from the infrared camera 120, for making frame rate adjustments to the video camera 122 image, and for combining the two images for superimposed display on the display 126.

The systems described above have numerous surgical applications. For example, the system may be deployed as an aid to cardiac surgery, where it may be used intraoperatively for direct visualization of cardiac blood flow, for direct visualization of myocardium at risk for infarction, and for image-guided placement of gene therapy and other medicinals to areas of interest. The system may be deployed as an aid to oncological surgery, where it may be used for direct visualization of tumor cells in a surgical field or for image-guided placement of gene therapy and other medicinals to an area of interest. The system may be deployed as an aid to general surgery for direct visualization of any function amenable to imaging with infrared fluorescent substances, including blood flow and tissue viability. In dermatology, the system may be used for sensitive detection of malignant cells or other skin conditions, and for non-surgical diagnosis of dermatological diseases using infrared ligands and/or antibodies.

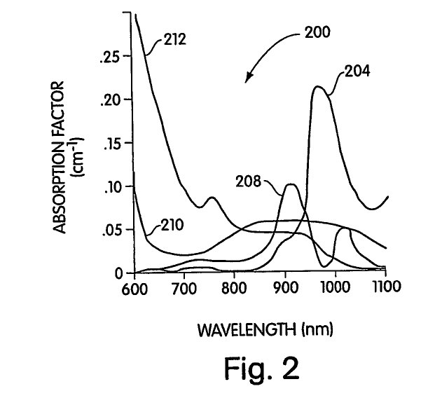

FIG. 2 shows an infrared window used by the imaging system. The infrared window 200 is characterized by wavelengths where absorbance is at a minimum. The components of living tissue with significant infrared absorbance include water 204, lipid 208, oxygenated hemoglobin 210, and deoxygenated hemoglobin 212. As shown in FIG. 2, oxygenated hemoglobin 210 and deoxygenated hemoglobin have significant absorbance below 700 nm. By contrast, lipids 208 and water 204 have significant absorbance above 900 nm. Between 700 nm and 900 nm, these absorbances reach a cumulative minimum referred to as the infrared window 200. While use of excitation and emission wavelengths outside the infrared window 200 is possible, as described in some of the examples above, infrared fluorescence imaging within the infrared window 200 offers several advantages including low tissue autofluorescence, minimized tissue scatter, and relatively deep penetration depths. While the infrared window 200 is one useful wavelength range for imaging, the systems described herein are not limited to either excitation or emission wavelengths in this window, and may employ, for example, far-red light wavelengths below the infrared window 200, or infrared light wavelengths above the infrared window 200, both of which may be captured using commercially available imaging equipment.

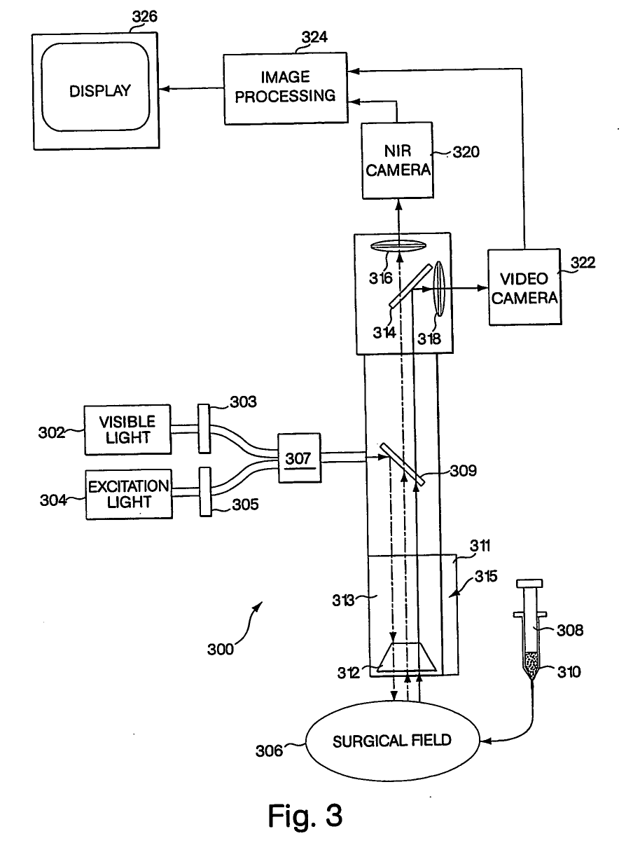

FIG. 3 shows an embodiment of an imaging system for use in an endoscopic tool. The imaging system 300 may include a visible light source 302, and excitation light source 304, a surgical field 306, a targeting moiety-conjugated infrared fluorescent substance source 308 containing a targeting moiety-conjugated infrared fluorescent substance 310 (reagent of the invention), a lens 312, a first filter 314, a second filter 316, a third filter 318, an infrared camera 320, a video camera 322, an image processing unit 324, and a display 326. In general, the visible light source 302 and the excitation light source 304 illuminate the surgical field 306. The targeting moiety-conjugated infrared fluorescent substance 310 may be introduced from the targeting moiety-conjugated infrared fluorescent substance source 308, such as through injection into the bloodstream of a subject. An image from the surgical field 306 is then captured by two cameras, the video camera 322 capturing a conventional, visible light image of the surgical field 306 and the infrared camera 320 capturing a diagnostic image based upon the distribution of the targeting moiety-conjugated infrared fluorescent substance 310 in the surgical field 306. These images may be combined by the image processing unit 324 and presented on a display 326 where they may be used, for example, by a surgeon conducting a surgical procedure. In general, each of these components may be any of those components similarly described with reference to FIG. 1 above. Differences for an endoscopic tool are now described.

The imaging system 300 for use as an endoscopic tool may further include a first lens/collimator 303 for the visible light source, a second lens/collimator 305 for the excitation light source 304, an optical coupler 307 that combines the excitation light and the visible light, a dichroic mirror 309, and an endoscope 311 having a first cavity 313 and a second cavity 315.

The first lens/collimator 303, the second lens/collimator 305, and the optical coupler 307 serve to combine the excitation light and the visible light into a single light source. This light source is coupled into the first cavity 313 through the dichroic mirror 309. In one embodiment, the dichroic mirror 309 preferably provides fifty percent reflection of light having wavelengths from 400 nm to 700 nm, in order to optimize an intensity of visible light that reaches the video camera 322 after illuminating the surgical field 306 and passing through the dichroic mirror 309 on its return path to the video camera 322. The dichroic mirror 309 also preferably has greater than ninety percent reflection of wavelength from the excitation light source 304, such as between 700 nm and 785 nm, so that these wavelengths are not transmitted to the cameras 320, 322 after reflecting off the surgical field. Using this arrangement, visible and excitation light sources 302, 304 share the first cavity 313 of the endoscope with the return light path for a visible light image and an emission wavelength image.

The second cavity 315 of the endoscope 311 may be provided for insertion of a tool, such as an optical tool like a laser for irradiation of a site in the surgical field 306, or a physical tool like an instrument for taking a biopsy of tissue within the surgical field. By combining the optical paths of the imaging system 300 within a single cavity of the endoscope 311, the combined gauge of the first cavity 313 for imaging and the second cavity 315 may be advantageously reduced.

The imaging system 300 may instead be used with a laparoscope. Typically, a laparoscope is inserted into a body cavity through an incision, as distinguished from an endoscope which is inserted through an existing body opening such as the throat or rectum. A laparoscope has a different form factor than an endoscope, including different dimensional requirements. Furthermore, use of a laparascope involves at least one additional step of making an incision into a body so that the laparascope may be inserted into a body cavity. The laparoscope may be used with any of the imaging systems described above, and the imaging system 300 of FIG. 3 in particular would provide the benefit of a narrower bore for illumination and imaging optics.

It will further be appreciated that the imaging system 300 may be used to simplify imaging devices other than endoscopes and laparoscopes, such as by providing an integrated, coaxial illumination and image capture device using the techniques described above.

In addition to the surgical applications noted above in reference to FIG. 1, the endoscopic tool of FIG. 3 may be used for direct visualization of malignant or pre-malignant areas within a body cavity, or for image-guided placement of gene therapy and other medicinals to an area of interest within the body cavity.

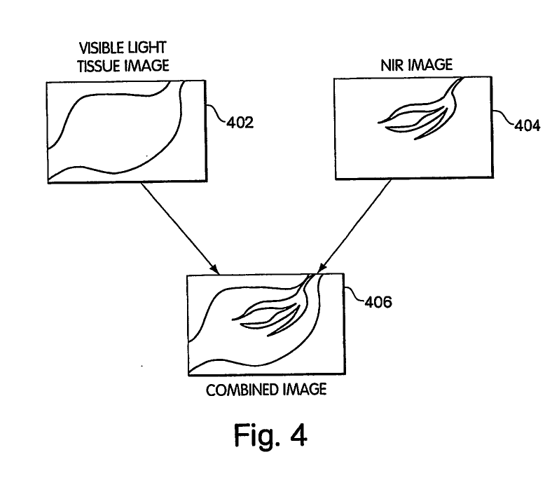

FIG. 4 shows an image displaying both a circulatory system and surrounding tissue. As described above, a visible light tissue image 402 is captured of tissue within a surgical field. As noted above, the visible light tissue image 402 may include a subset of visible light wavelengths when an optical channel for substance imaging includes a wavelength within the visible light range. An infrared image 404 is also captured of the same (or an overlapping) field of view of the surgical field. Although referred to here for convenience as an infrared image, it should be clear that the target moiety-conjugated infrared fluorescent substance-based image 404 may also, or instead, employ other wavelengths, such as far-red or infrared wavelengths. The infrared image 404 may be shifted to a visible wavelength for display, preferably using a color that is prominent when superimposed on the visible light tissue image 402. The images 402, 404 may be frame-rate adjusted as appropriate for video display of the surgical field.

The images may be displayed separately as the visible light tissue image 402 and the infrared image 404. Or the images 402, 404 may be combined into a combined image 406 by the image processing unit described above. The combined image 406 may then be used as an aid to the procedures described above, or to any other surgical or diagnostic procedure that might benefit from the substance-based imaging techniques described herein.

The disclosure of U.S. application 60/363,413 is hereby incorporated by reference in its entirety.

### EXAMPLE 1

### Preparation of Annexin Conjugate

The N-hydroxysuccinimide (NHS) ester of IRDye78 (IRDye78-NHS) was purchased from LI-COR (Lincoln, Nebr.) and stored desiccated, under nitrogen, at −80° C. Recombinant human Annexin V was purchased from BDPharmingen (Catalog #556416; 100 μg in PBS at 1 mg/ml). The protein is preferably provided in a non-amine-containing buffer such as PBS. The conjugation reaction is less successful in Tris or other amine-containing buffers.

In general, NHS ester labelings in aqueous solution should be performed in the presence of an excess of NHS ester to nucleophile. The half-life of NHS esters in aqueous buffers at pH 7.4 is rather short. Therefore, one must typically use a high molar ratio of fluorophore to protein since water itself will compete for hydrolysis of the NHS ester. Human Annexin V has the following nucleophiles:

| | |
|---|---|
| Total (27 nucleophiles): | 1 alpha amine |
| | 22 Lysines |
| | 1 Cysteine |
| | 3 Histidine |

In the subsequent experiments, Annexin V was provided as a solution (0.5 mg/ml; MW 36,000, 14 μM protein, 375 μM in nucleophiles). The molarity in parentheses below represents, with respect to Annexin, the hypothetical concentration of nucleophiles in the final reaction solution, rather than the final concentration of Annexin itself. IRDye78-NHS was provided as a 13 mM solution in DMSO.

| Test Labelings: | 2:1 Ratio | 7:1 Ratio | 20:1 Ratio |
|---|---|---|---|
| Annexin V | 5 μL | 5 μL | 5 μL |
| | (188 μM) | (188 μM) | (188 μM) |
| IRDye78-NHS | 0.33 μL | 1 μL | 2.9 μL |
| | (0.43 mM) | (1.3 mM) | (3.8 mM) |
| PBS, pH 7.4 | 4.7 μL | 4 L | 2.12 μL |
| Total volume | 10 μL | 10 μL | 10 μL |

The reagents were mixed adding the NHS ester last, and the mixture was immediately vortexed continuously 30 min at room temperature at low speed to avoid frothing. The reaction was quenched by adding 1 μL of 1 M Tris, pH 8.0 (100 mM final), vortex, and incubated without vortexing 15 minutes. Bound dye was separated from unbound dye using a Vivaspin column (see below).

### EXAMPLE 2

### Vivaspin Filter Purification

A 10,000 M.W. Vivaspin 500 filter with a low protein binding polyethersulfone membrane from Vivascience (Cat. No. VS0101) was used. Maximum g force is 15,000.

The filter was pre-washed with 200 μL of PBS, vortexed at 15,000×g for 15 minutes, and the solution discarded.

The Annexin V sample was diluted with PBS to a final volume of 500 μL and placed in the sample chamber. The combination was vortexed at 15,000×g for 20 minutes. 5 μL of retentate remained in the top chamber.

The elutate was discarded, and 300 μL of PBS was added and vortexed at 15,000×g for 15 minutes. About 5 μL retentate remained in the top chamber. This step was then repeated.

35 μL PBS was used to wash membrane and recover labeled protein. A preference has been observed for Annexin V to bear a single IRDye 78 label, even in the presence of an excess of labelling reagent.

### EXAMPLE 3

### Cell-Based Assay

U937 cells were grown in suspension (Medium: RPMI+10% heat-inactivated FBS). 1 mL of the suspension was incubated with 1.4 nM TNF-α for 90 min. at 37° C.

Conjugation using a 2:1 ratio of annexin to dye was performed during incubation. A filter was pre-washed with 200 μL of HBSS, vortexed at 15,000×g for 15 minutes, and discarded. The conjugated Annexin sample was diluted with HBSS to a final volume of 500 μL and placed in the sample chamber. The combination was vortexed at 15,000×g for 20 min, leaving ≈5 μL of retentate in the top chamber. The elutate was discarded, and 300 μL HBSS was added and vortexed at 15,000×g for 15 minutes, leaving ≈5 μL of retentate in the top chamber. 95 μL of HBSS was added and used to wash the membrane and recover the labeled protein. After incubation, the cells were spun at 1000×g for 1 min., the media was aspirated, and the remainder washed 2× at 1000×g for 1 min with HBSS after last wash. The cells were then resuspended with 50 μL of HBSS/1% NFD (non-fat dry milk). Blocking with 0.5% NFD (non-fat dry) milk is helpful to reduce background radiation.

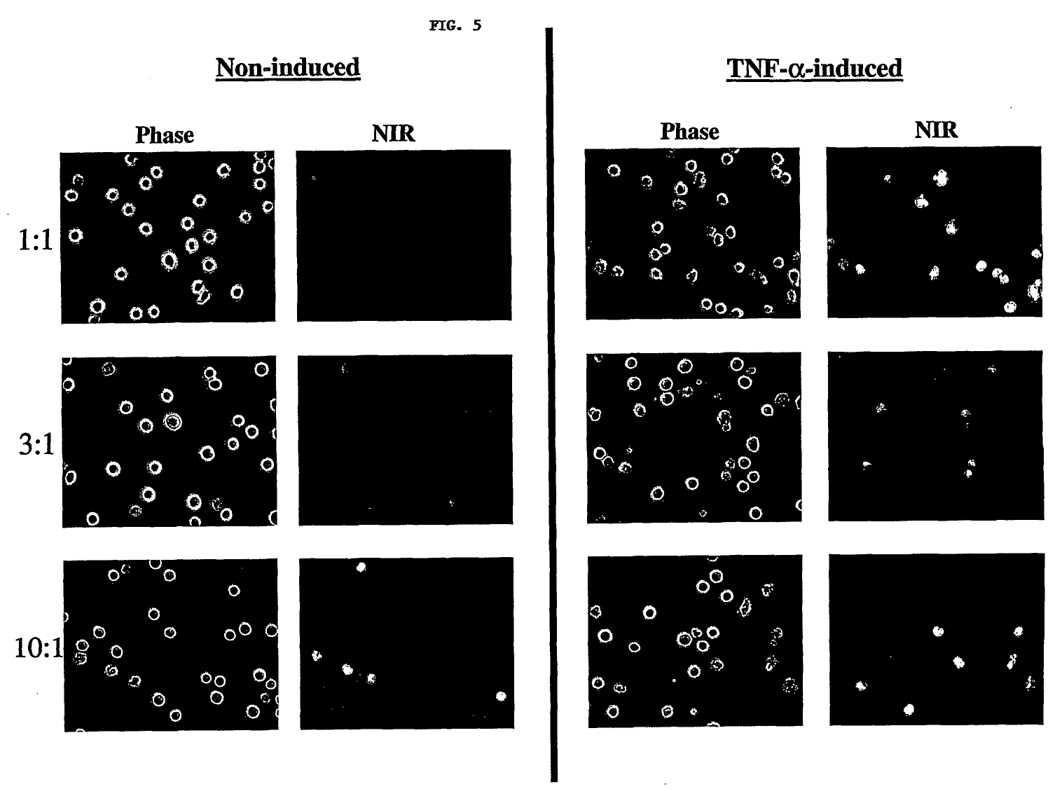

50 μL of the conjugation solution was added to the cells and the mixture was incubated at RT for 30 min, washed 2× with 1 mL HBSS, and resuspended in 500 μL HBSS. 5 μL of cell suspension was removed and placed on a slide for observation, or the entire solution was tested by flow cytometry. Results of an exemplary assay are depicted in FIG. 5.

Reagents:

Recombinant Human TNF-α (R&D Systems 210-TA, 10 μg vial; 17.5 kDa). Resuspend powder in PBS/0.1% BSA (final concentration=1.4 μM), flash freeze and store at −80° C.

Annexin V-FITC (BD Pharmingen 556419, 200 tests/vial, 1 mL vial) store at 4° C.

Hanks balanced salt solution (HBSS), 1× without phenol red (Fisher Scientific BW10-527F, 500 mL) store at 4° C.

### EXAMPLE 4

### Red Blood Cell-Based Assay

100 μL of whole blood was spun down at 500×g for 1 minute, then the serum was aspirated and 500 μL of PBS0.5% NFD Milk was added. The mixture was re-spun at 500×g for 1 minute, aspirated, and resuspended in 500 μL of PBS+0.5% NFD Milk. The 40 μL of each Annexin test reaction (or 40 μL of PBS control) was diluted with 40 μL of (PBS+2 mM CaCl₂) then split into 2 tubes (40 μL per tube). To one of the tubes, 1 μL of calcium ionophore A23187 from a 0.2 mM stock solution was added (final A23187=5 μM). The solutions for were incubated for 45 min at 37° C. in a water bath. The solution was subjected to cytospin at 300 rpm for 10 minutes and viewed under near-infrared fluorescence.

Reagents:

Calcium ionophore A23187 (Sigma #C-7522; 10 mg vial; MW 523). Resuspend powder in 1.91 ml of DMSO (final concentration=10 mM). To make 0.2 mM stock solutions dilute 1:50 in DMSO. Store at −20° C.

### EXAMPLE 5

### Image Displaying Near-Infrared Fluorescence Imaging of Annexin V In Vivo

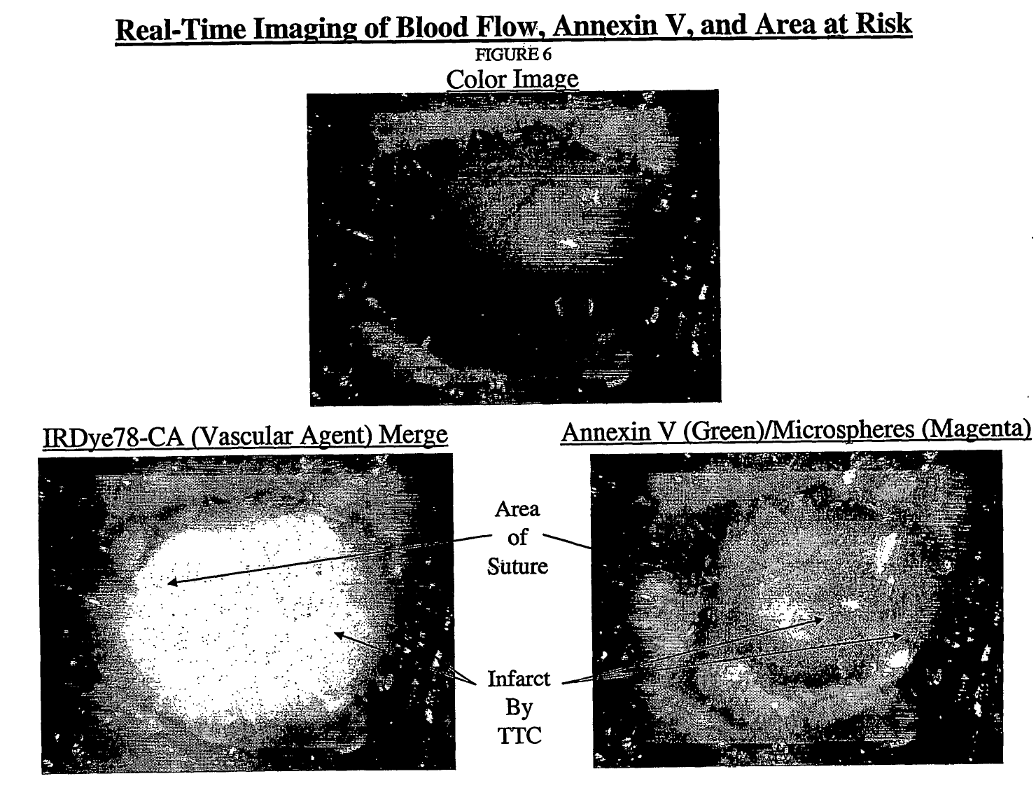

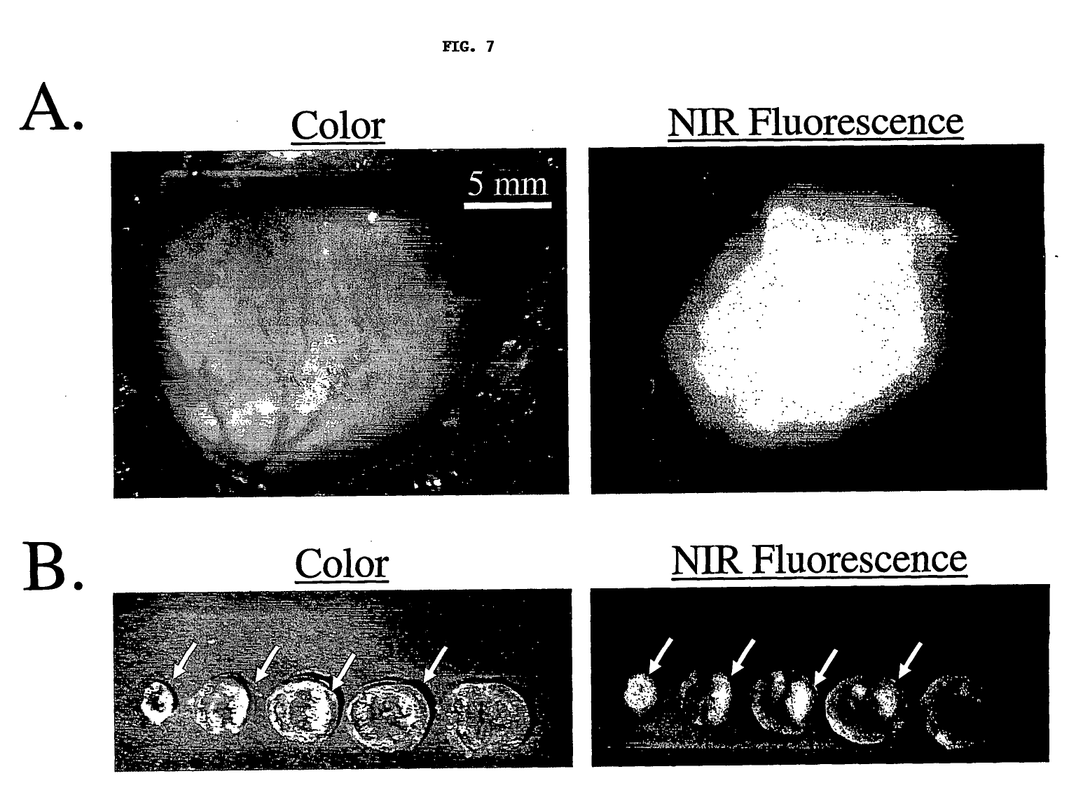

A branch of the left anterior descending (LAD) artery of the rat heart was ligated with a silk suture. During ligation, the aorta was cross-clamped and 10 μm red fluorescent microbeads were injected into the ventricle. These beads mark the location of blood flow during ischemia, and their red fluorescence does not interfere with the near-infrared fluorescence from the annexin V covalently conjugated with IRDye78. After 20 minutes of ligation, the suture was removed, and the heart was permitted to re-perfuse. 24 hours after LAD artery ischemia, the beating rat heart was imaged, as shown in FIGS. 6 and 7, using the intraoperative near-infrared fluorescence imaging system.

The top panel of FIG. 6 shows the color video image. The lower left panel of FIG. 6 shows the vasculature of the heart (in white) as delineated by injection of 12.5 nmol indocyanine green, a near-infrared fluorescent vascular contrast agent. The otherwise invisible near-infrared fluorescence from indocyanine green has been pseudo-colored white prior to superimposition on the color video (anatomic) image. Indocyanine green staining reveals an area of avascularity that corresponds to infarcted tissue when this heart was subsequently stained with 2,3,5-triphenyltetrazolium chloride. The lower right image of FIG. 6 shows the superimposition of three separately acquired frames. The color video image shows the anatomy of the heart. In pseudo-colored magenta are the red fluorescent microbeads injected during initial ischemia. This area was well perfused during ischemia. The area of the heart internal to this “ring” of perfusion is termed the “area at risk”, i.e., the area of myocardium that had no blood flow during LAD ligation. The area of avascularity/infarct identified with indocyanine green is contained within the area at risk. Finally, the near-infrared fluorescence signal from injection of 25 μg of human annexin V covalently conjugated with IRDye78 (a near-infrared fluorophore) is shown pseudo-colored in green. The green areas, thus, represent apoptotic and/or necrotic cells, i.e., those areas within the area at risk that have actually succumbed to the ischemia. As can be seen, there are several areas of infarcted myocardium within the area at risk, and distinct from already infarcted and avascular myocardium, that are not otherwise apparent from either the color video or vascular images. Also, a band-like area in the lower portion of the picture, which is immediately above well-perfused myocardium, shows no cell death. Presumably, this area was supplied by either diffusion or smaller collateral vessels fed by the well-perfused area.

FIG. 7A shows another view of a labelled ischemic heart in situ, and FIG. 7B shows a series of sections of such a heart, further exemplifying the utility of the labelled annexin V in identifying the injured tissue.

It will be appreciated that the above functionality is merely illustrative, and that other dyes and substances, imaging hardware, and optics may be usefully deployed with the imaging systems described herein. For example, an endoscopic tool may employ a still-image imaging system for diagnostic photography within a body cavity. Or any of the imaging systems may be used as described above with excitation and/or emission wavelengths in the far-red spectrum. Through minor adaptations that would be clear to one of ordinary skill in the art, the system could be configured to image two or more functions (i.e., tumor and blood flow) at the same time that a visible light image is captured by associating each function with a different substance having a different emission wavelength. These and other arrangements and adaptations of the subject matter discussed herein are intended to fall within the scope of the invention.

Thus, while the invention has been disclosed in connection with the preferred embodiments shown and described in detail, various modifications and improvements thereon will become readily apparent to those skilled in the art. It should be understood that all matter contained in the above description or shown in the accompanying drawings shall be interpreted as illustrative, and not in a limiting sense, and that the following claims should be interpreted in the broadest sense allowable by law.

All references, patents, and patent applications cited herein are hereby incorporated by reference in their entireties.

1. A reagent comprising a targeting moiety that selectively localizes to cells or tissue undergoing cell death, the targeting moiety being covalently conjugated to an infrared fluorescent substance.

2. The reagent according to claim 1, wherein the infrared fluorescent substance is a near-infrared fluorescent dye having a structure of the formula: 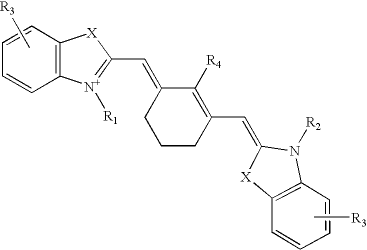
wherein, as valence and stability permit,
X represents C(R)₂, S, Se, O, or NR₅;
R represents H or lower alkyl, or two occurrences of R, taken together, form a ring together with the carbon atoms through which they are connected;
R₁ and R₂ represent, independently, substituted or unsubstituted lower alkyl, lower alkenyl, cycloalkyl, cycloalkylalkyl, aryl, or aralkyl, optionally substituted by sulfate, phosphate, sulfonate, phosphonate, halogen, hydroxyl, amino, cyano, nitro, carboxylic acid, or amide, or a pharmaceutically acceptable salt thereof;
R₃ represents, independently for each occurrence, one or more substituents to the ring to which it is attached, such as a fused ring, sulfate, phosphate, sulfonate, phosphonate, halogen, lower alkyl, hydroxyl, amino, cyano, nitro, carboxylic acid, or amide, or a pharmaceutically acceptable salt thereof;
R₄ represents H, halogen, or a substituted or unsubstituted ether or thioether of phenol or thiophenol; and
R₅ represents, independently for each occurrence, substituted or unsubstituted lower alkyl, cycloalkyl, cycloalkylalkyl, aryl, or aralkyl, optionally substituted by sulfate, phosphate, sulfonate, phosphonate, halogen, hydroxyl, amino, cyano, nitro, carboxylic acid, amide, etc., or a pharmaceutically acceptable salt thereof.

3. A method of imaging cell death comprising
(a) contacting a sample of cells with a reagent of claim 1,
(b) positioning the sample adjacent to an electronic imaging device, and
(c) constructing an image of emission wavelength,
wherein said image is a representation of cell death in said sample.

4. A pharmaceutical preparation comprising a reagent of claim 1 and a pharmaceutically acceptable excipient.

5. A composition comprising a reagent of claim 1.

6. The reagent according to claim 1, wherein the targeting moiety is a protein or a fragment thereof.

7. The reagent according to claim 1, wherein the targeting moiety is a protein, or a fragment thereof, having an amino acid sequence at least 60% homologous to the amino acid sequence of an annexin protein.

8. The reagent according to claim 7, wherein the protein, or fragment thereof, has at least one biological activity of an annexin protein, such as having a high affinity for anionic phospholipids surfaces.

9. The reagent according to claim 1, wherein the targeting moiety is annexin V.

10. The reagent according to claim 1, wherein the fluorescent substance is a quantum dot.

11. The reagent according to claim 10, wherein the infrared fluorescent substance is a quantum dot and the targeting moiety is annexin V.

12. The reagent according to claim 1, wherein the infrared fluorescent substance is iodocyanine green, IRDye78, IRDye80, IRDye38, IRDye40, IRDye41, IRDye700, IRDye800, Cy7, IR-786, DRAQ5NO, or analogs thereof.

13. The reagent according to claim 12, wherein the targeting moiety is annexin V and the infrared fluorescent substance is IRDye78 or an analog thereof.

14. The reagent according to claim 1, wherein the infrared fluorescent substance/targeting moiety conjugate is purified from unconjugated infrared fluorescent substance through gel filtration or dialysis.

15. The reagent according to claim 1, or 6, wherein the infrared fluorescent substance/targeting moiety conjugate is soluble in blood.

16. The reagent according to claim 1, wherein the infrared fluorescent substance/targeting moiety conjugate is used to image organ and bone marrow transplant rejection or injury, infectious and non-infectious inflammatory diseases, autoimmune disease, cerebral and myocardial infarction and ischemia, cardiomyopathies, atherosclerotic disease, neural and neuromuscular degenerative diseases, sickle cell disease, β-thalassemia, cancer therapy, AIDS, myelodysplastic syndromes, such as aplastic anemia, toxin-induced liver disease, traumatic injury, bacterial infection, or acute hypoxia.

17. The reagent according to claim 1, wherein the infrared fluorescent substance/targeting moiety conjugate is used to image ischemic injury.

18. The reagent according to claim 17, wherein the ischemic injury is myocardial infarction, reperfusion injury or stroke.

19. A method of imaging cell death within a region in vivo, comprising
(a) administering to a subject a preparation of claim 4,
(b) positioning the region adjacent to an electronic imaging device, and
(c) constructing an image of emission wavelength,
wherein said image is a representation of cell death within a region of said subject.

20. The method according to claim 19, wherein the electronic imaging device captures an image of a field of view that includes some portion of the subject, the image including a first image obtained from the one or more wavelengths of visible light and a second image obtained from the emission wavelength.

21. The method according to claim 19, wherein the electronic imaging device captures a visible light image of the surgical field and an emission wavelength image of the surgical field.

22. The method according to claim 19, wherein the electronic imaging device captures an image of a field of view that includes some portion of the subject, the image including a first image obtained from the one or more wavelengths of visible light and a second image concurrently obtained from the emission wavelength.

23. The method according to claim 19, wherein the preparation is administered intravenously, intraperitoneally, intrathecally, intrapleurally, intralymphatically, intravaginally, intravesically, intrarectally, or intramuscularly.

24. The method according to claim 19, wherein the preparation is topically applied to the region.

25. The method according to claim 19, wherein the preparation is administered in a dose of less than 300 μg protein/kg.

26. The method according to claim 19, wherein the preparation is administered in a dose of less than 50 μg protein/kg.

27. The method according to claim 19, wherein the preparation is administered in a dose of less than 10 μg protein/kg.

28. A method for detecting cell death in a cell sample or tissue sample, comprising
(a) treating the sample with a reagent of claim 1,
(b) irradiating the sample with a light source,
(c) detecting an emission wavelength of the infrared fluorescent substance.

29. The reagent according to claim 1, wherein the fluorescent substance will have an emission wavelength in a range from about 680 nm to about 100,000 nm.

30. The reagent according to claim 1, wherein the fluorescent substance will have an emission wavelength in a range from about 680 nm to about 20,000 nm,

31. The reagent according to claim 1, wherein the fluorescent substance will have an emission wavelength in a range from about 700 nm to about 1,000 nm.

32. The reagent according to claim 2, wherein two occurrences of R taken together form a six-membered ring.

33. The reagent according to claim 1, wherein the infrared fluorescent substance is a near-infrared fluorescent dye having a structure of the formula: 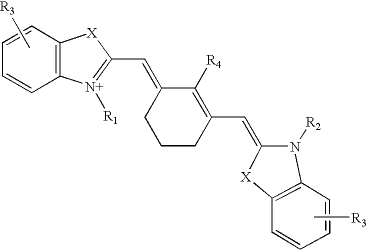
wherein, as valence and stability permit,
X represents C(R)₂, S, Se, O, or NR₅;
R₁ and R₂ represent, independently, substituted or unsubstituted lower alkyl, lower alkenyl, cycloalkyl, cycloalkylalkyl, aryl, or aralkyl, optionally substituted by sulfate, phosphate, sulfonate, phosphonate, halogen, hydroxyl, amino, cyano, nitro, carboxylic acid, or amide, or a pharmaceutically acceptable salt thereof;
R₃ represents, independently for each occurrence, one or more substituents to the ring to which it is attached, such as a fused ring, sulfate, phosphate, sulfonate, phosphonate, halogen, lower alkyl, hydroxyl, amino, cyano, nitro, carboxylic acid, or amide, or a pharmaceutically acceptable salt thereof;
R₄ represents H, halogen, or a substituted or unsubstituted ether or thioether of phenol or thiophenol; and
R₅ represents, independently for each occurrence, substituted or unsubstituted lower alkyl, cycloalkyl, cycloalkylalkyl, aryl, or aralkyl, optionally substituted by sulfate, phosphate, sulfonate, phosphonate, halogen, hydroxyl, amino, cyano, nitro, carboxylic acid, or amide, or a pharmaceutically acceptable salt thereof.

34. The reagent according to claim 32, wherein the near-infrared fluorescent dye is selected from IRDye78, IRDye80, IRDye38, IRDye40, IRDye41, IRDye700, IRDye800, Cy7, and compounds formed by conjugating a second molecule to any of IRDye78, IRDye80, IRDye38, IRDye40, IRDye41, IRDye700, IRDye800, and Cy7.
